SEQUENCE LISTING (32 sequences)

SEQ ID NO: 1 - protein, 610 aa, Homo sapiens
  misc_feature: Incyte clone 044150
MAAPEAWRARSCWFCEVAAATTMEATSREAAPAKSSASGPNAPPALFELCGRAVSAHMGVLESGVWALPGPILQSILPLLNIYYLERIEETALKKGLSTQAIWRRLWDELMKTRPSSLESVTCWRAKFMEAFFSHVLRGTIDVSSDRRLCDQRFSPSAPAATSSASSSTSSYKRAPASSAPQPKPLKRFKRAAGKKGARTRQGPGAESEDLYDFVFIVAGEKEDGEEMEIGEVACGALDGSDPSCLGLPALEASQRFRSISTLELFTVPLSTEAALTLCHLLSSWVSLESLTLSYNGLGSNIFRLLDSLRALSGQAGCRLRALHLSDLFSPLPILELTRAIVRALPLLRVLSIRVDHPSQRDNPGVPGNAGPPSHIIGDEEIPENCLEQLEMGFPRGAQPAPLLCSVLKASGSLQQLSLDSATFASPQDFGLVLQTLKEYNLALKRLSFHDMNLADCQSEVLFLLQNLTLQEITFSFCRLFEKRPAQFLPEMVAAMKGNSTLKGLRLPGNRLGNAGLLALADVFSEDSSSSLCQLDISSNCIKPDGLLEFAKRLERWGRGAFGHLRLFQNWLDQDAVTAREAIRRLRATCHVVSDSWDSSQAFADYVSTM

SEQ ID NO: 2 - protein, 275 aa, Homo sapiens
  misc_feature: Incyte clone 266775
MEAKTRELIARRTTPLLEYIKNRKLEKQRIREEKREERRRRELEKKRLREEEKRRRREEERCKKKETDKQKKIAEKEVRIKLLKKPEKGEEPTTEKPKERGEEIDTGGGKQESCAPGAVVKARPMEGSLEEPQETSHSGSDKEHRDVERSQEQESEAQRYHVDDGRRHRAHHEPERLSRRSEDEQRWGKGPGQDRGKKGSQDSGAPGEAMERLGRAQRCDDSPAPRKERLANKDRPALQLYDPGARFRARECGGNRRICKAEGSGTGPEKREEAE

SEQ ID NO: 3 - protein, 147 aa, Homo sapiens [sequence omitted]

SEQ ID NO: 4 - protein, 336 aa, Homo sapiens [sequence omitted]

SEQ ID NO: 5 - protein, 213 aa, Homo sapiens
  misc_feature: Incyte clone 1441620
MAITQFRLFKFCTCLATVFSFLKRLICRSGRGRKLSGDQITLPTTVDYSSVPKQTDVEEWTSWDEDAPTSVKIEGGNGNVATQQNSLEQLEPDYFKDMTPTIRKTQKIVIKKREPLNFGIPDGSTGFSSRLAATQDLPFIHQSSELGDLDTWQENTNAWEEEEDAAWQAEEVLRQQKLADREKRAAEQQRKKMEKEAQRLMKKEQNKIGVKLS

SEQ ID NO: 6 - protein, 338 aa, Homo sapiens
  misc_feature: Incyte clone 1510911
MTALSSENCSFQYQLRQTNQPLDVNYLLFLIILGKILLNILTLGMRRKNTCQNFMEYFCISLAFVDLLLLVNISIILYFRDFVLLSIRFTKYHICLFTQIISFTYGFLHYPVFLTACIDYCLNFSKTTKLSFKCQKLFYFFTVILIWISVLAYVLGDPAIYQSLKAQNAYSRHCPFYVSIQSYWLSFFMVMILFVAFITCWEEVTTLVQAIRITSYMNETILYFPFSSHSSYTVRSKKIFLSKLIVCFLSTWLPFVLLQVIIVLLKVQIPAYIEMNIPWLYFVNSFLIATVYWFNCHKLNLKDIGLPLDPFVNWKCCFIPLTIPNLEQIEKPISIMIC

SEQ ID NO: 7 - protein, 326 aa, Homo sapiens [sequence omitted]

SEQ ID NO: 8 - protein, 529 aa, Homo sapiens
  misc_feature: Incyte clone 2024312
MLVLFETSVGYAIFKVLNEKKLQEVDSLWKEFETPEKANKIVKLKHFEKFQDTAEALAAFTALMEGKINKQLKKVLKKIVKEAHEPLAVADAKLGGVIKEKLNLSCIHSPVVNELMRGIRSQMDGLIPGVEPREMAAMCLGLAHSLSRYRLKFSADKVDTMIVQAISLLDDLDKELNNYIMRCREWYGWHFPELGKIISDNLTYCKCLQKVGDRKNYASAKLSELLPEEVEAEVKAAAEISMGTEVSEEDICNILHLCTQVIEISEYRTQLYEYLQNRMMAIAPNVTVMVGELVGARLIAHAGSLLNLAKHAASTVQILGAEKALFRALKSRRDTPKYGLIYHASLVGQTSPKHKGKISRMLAAKTVLAIRYDAFGEDSSSAMGVENRAKLEARLRTLEDRGIRKISGTGKALAKTEKYEHKSEVKTYDPSGDSTLPTCSKKRKIEQVDKEDEITEKKAKKAKIKVKVEEEEEEKVAEEEETSVKKKKKRGKKKHIKEEPLSEEEPCTSTAIASPEKKKKKKKKRENED

SEQ ID NO: 9 - protein, 361 aa, Homo sapiens
  misc_feature: Incyte clone 2057886
MRGQRSLLLGPARLCLRLLLLLGYRRRCPPLLRGLVQRWRYGKVCLRSLLYNSFGGSDTAVDAAFEPVYWLVDNVIRWFGVVFVVLVIVLTGSIVAIAYLCVLPLILRTYSVPRLCWHFFYSHWNLILIVFHYYQAITTPPGYPPQGRNDIATVSICKKCIYPKPARTHHCSICNRCVLKMDHHCPWLNNCVGHYNHRYFFSFCFFMTLGCVYCSYGSWDLFREAYAAIETYHQTPPPTFSFRERMTHKSLVYLWFLCSSVALALGALTVWHAVLISRGETSIERHINKKERRRLQAKGRVFRNPYNYGCLDNWKVFLGVDTGRHWLTRVLLPSSHLPHGNGMSWEPPPWVTAHSASVMAV

SEQ ID NO: 10 - protein, 361 aa, Homo sapiens
  misc_feature: Incyte clone 2121924
MFAKGKGSAVPSDGQAREKLALYVYEYLLHVGAQKSAQTFLSEIRWEKNITLGEPPGFLHSWWCVFWDLYCAAPERRDTCEHSSEAKAFHDYSAAAAPSPVLGNIPPNDGMPGGPIPPGFFQPFMSPRYAGGPRPPIRMGNQPPGGVPGTQPLLPNSMDPTRQQGHPNMGGSMQRMNPPRGMGPMGPGPQNYGSGMRPPPNSLGPAMPGINMGPGAGRPWPNPNSANSIPYSSSSPGTYVGPPGGGGPPGTPIMPSPADSTNSSDNIYTMINPVPPGGSRSNFPMGPGSDGPMGGMGGMEPHHMNGSLGSGDIDGLPKNSPNNISGISNPPGTPRDDGELGGNFLHSFQNDNYSPSMTMSV

SEQ ID NO: 11 - protein, 221 aa, Homo sapiens
  misc_feature: Incyte clone 2122815
MRGLHPWHVLRRPLGPQAHANDPECGQRPVPALSHHGSQRVVLLQTATLLGVLLLGYGYFWLLVPNPEARLQQLGLFCSVFTISMYLSPLADLAKVIQTKSTQCLSYPLTIATLLTSASWCLYGFRLRDPYIMVSNFPGIVTSFIRFWLFWKYPRSKTGTTGSCKPEAAHLTTGHLSANLNQRDLLVSAGPAVQLPRCSGLWEQEMTLRIKGPKKKLYLDD

SEQ ID NO: 12 - protein, 238 aa, Homo sapiens
  misc_feature: Incyte clone 2132179
MALVPCQVLRMAILLSYCSILCNYKAIEMPSHQTYGGSWKFLTFIDLVIQAVFFGICVLTDLSSLLTRGSGNQEQERQLKKLISLRDWMLAVLAFPVGVFVVAVFWIIYAYDREMIYPKLLDNFIPGWLNHGMHTTVLPFILIEMRTSHHQYPSRSSGLTAICTFSVGYILWVCWVHHVTGMWVYPFLEHIGPGARIIFFGSTTILMNFLYLLGEVLNNYIWDTQKSMEEEKEKPKLE

SEQ ID NO: 13 - protein, 348 aa, Homo sapiens
  misc_feature: Incyte clone 2326441
MGAACPLSSPVYSTPPPWLWPWPTSMGPGSGRGTTSCATPVTAASWLAPASMLACPQRNPSTSAAGPRIMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQHRTRGSCPRADGARREVLPDKL

SEQ ID NO: 14 - protein, 352 aa, Homo sapiens
  unsure 320: unknown, or other
MSMLAERRRKQKWAVDPQNTAWSNDDSKFGQRMLEKMGWSKGKGLGAQEQGATDHIKVQVKNNHLGLGATINNEDNWIAHQDDFNQLLAELNTCHGQETTDSSDKKEKKSFSLEEKSKISKNRVHYMKFTKGKDLSSRSKTDLDCIFGKRQSKKTPEGDASPSTPEENETTTTSAFTIQEYFAKRMAALKNKPQVPVPGSDISETQVERKRGKKRNKEATGKDVESYLQPKAKRHTEGKPERAEAQERVAKKKSAPAEEQLRGPCWDQSSKASAQDAGDHVQPPEGRDFTLKPKKRRGKKKLQKPVEIAEDATLEETLVXKEEEERFQMNPSQPGPSDHSAVRALRGQTPLA

SEQ ID NO: 15 - protein, 210 aa, Homo sapiens [sequence omitted]

SEQ ID NO: 16 - protein, 318 aa, Homo sapiens [sequence omitted]

SEQ ID NO: 17 - DNA, 2316 bp, Homo sapiens
  misc_feature: Incyte clone 044150
cacctccccaagatggcggcgcccgaggcctggcgcgcccggagttgctggttctgtgaggtagcggcggcaacgaccatggaggccacgtcccgggaggcggcgccagcgaagagctcggcctcgggccccaacgctccccccgccctgttcgagctgtgcgggcgggcggtgagcgcccatatgggggttctggagagcggggtgtgggccctcccaggcccaatacttcaaagcatcctacctctgctcaatatatattacttggagaggattgaggaaactgccctcaagaaaggcctctcaactcaggccatctggcgccgactctgggatgaactgatgaagacaaggccttccagtttggaaagtgtgacatgttggcgagccaagtttatggaggcctttttttcccatgttctacgtgggaccattgatgtgtcttctgacaggcgtctttgtgatcagcggttctcaccttctgctccagcagccacctcctctgcctcttcttctacatcctcatacaaacgggcaccagctagctcagccccacagcctaagcccctaaagcgtttcaagcgagctgcagggaagaagggtgctcgcacccgtcaggggcctggtgcagagtctgaagacctgtatgacttcgtttttattgtggctggcgagaaggaggatggcgaagagatggagattggggaagtggcttgtggagctttggatggatcagatcccagctgcctggggcttccagcactggaagcttcacaaagattccgcagcatctccaccttggagctattcacagttccactctccacagaggcagccctgacactatgccacctgctgagctcctgggtgtcactggagagcctcacactctcctacaatggcctgggctctaacatcttccgcctgctagacagcctgcgggccctgtcaggccaggctggatgtcgcctccgtgccctgcatctcagtgacctgttctcaccactgcccatcctggagctgacacgtgctatcgtgcgagcactgcccctgctacgggtcctctctattcgtgttgaccacccaagccagcgggacaaccctggtgtgccagggaatgcagggccccctagccacataataggcgatgaggagataccagaaaactgcctggagcagttggagatgggatttccacggggagcccagccagccccactgctgtgctccgttctgaaggcctcgggttctctgcagcagctgtccctggatagtgccacctttgcctctccccaggattttgggcttgttttgcaaacactcaaagagtacaacctagccctgaaaagactgagcttccatgacatgaatctcgctgactgtcagagcgaggtgctctttttgctacagaatctgactctgcaagagattaccttctccttctgccgtctgtttgagaagcgcccagcccaatttctgcctgagatggttgctgctatgaagggcaactccacactgaagggcctccggctgccagggaaccgcctggggaatgctggcctgctggccttggcagatgttttctcagaggattcatcctcctctctctgtcagctggacatcagttccaactgcatcaagccagatgggcttctggagttcgccaagcggctggagcgctggggccgtggagcctttggtcacctgcgcctcttccaaaactggctggaccaggatgcagtcacagccagggaagccatccggcggctccgggctacctgccatgtggttagcgactcatgggactcatcccaggccttcgcagattatgttagcaccatgtgatggggcccgtacctcacagtctcatgctcggtaccatcagcttgcaggggctgaagcatgggctgcccagaaccccaaccaccagttctatctttctctttctgtcaccttttttctcttttttccttcttcccttgcactgaggtcctggaggccttgatgaggcccagcaaacaggcattctcacagctgggtttatagtctttgggccccttactcagtatcctgggaaccctgggccaggaggttacagtggtcatcataattgctgaagagatcccctcccctgcccctgggttcctgccttccctcctcaagcaggcacccaggctttagagaagtatagggggcttcttccctgctgggcttaccacactgctctcaggcctcaaaccctttcatacctttattcttttttttaaccaaaaaagtttttcttataaaataaattttgggcaaacaaaaaaaaaa

SEQ ID NO: 18 - DNA, 2569 bp, Homo sapiens
  misc_feature: Incyte clone 266775
gaaaaataccaccaacaaagaaacacagatgtggtttacatgaaaaatgcggatgattttcagacggctaacgcttaggaaagcgggtgcctttgaaaggaccagcgttgcccgcccggcgcctcccgggcttccctgctcgtccgcggacggggcgctgcggggccggggggcgccggctcttcctgtggcctccacgctggtgccgcagccagtgcggttttaaataccggagaaggtccccaagtcaggagagtctctcggcgccacgggttcctctgggagtgcgccctggccttgccttagggtttcagcctcggaggaccggttctgggcagtggagaagggacctgagttctgccttgtaaagttaacgttttgcgtttgtttttgctaaagaatatccaagttgttacaattaactgagatgatttggcacaaaagttttatctaaagtagtttgttgtgcccagaaaaggaaaaagaggctaaattaatggactattgtatttttcactgaccattttcactgttatctcttatttcagtctttatcctcatctctactcaagagcatacattaattttaggaatcctgatgacatccttctttttagagatcgttttgatggatatatcttccttgacagcaaaggcctagaatatcctgcagtggtagagtttgctccattccagaagatagccaaaaagaagctgagaaaaaaagatgccaagactggaagcatcgaagatgatccagaatataagaagtttttagaaacctactgtgtggaggaagagaagaccagtgccaaccctgagactctgctgggggagatggaggcgaagacaagagagctcattgctagaagaaccacacctcttttggaatatattaaaaatagaaaattagaaaagcagagaattcgagaagagaagcgagaagaacggaggaggagagagttagaaaagaaacgtttgcgggaagaggaaaaaagaagaagaagagaagaagaaagatgcaaaaaaaaagagacagataaacagaagaaaattgcagagaaagaagtaaggattaagcttcttaagaaaccagaaaagggagaggaaccaaccacagagaaaccaaaagaaagaggagaggagattgatactggaggtggcaagcaggaatcctgtgcccccggtgcagtcgtaaaagccaggcccatggaaggctcgctggaggagccccaggagacgtcacacagcggcagtgataaagagcacagggatgtggagagatctcaagaacaagaatctgaagcacaaagataccatgtggatgacggcaggaggcacagagctcaccacgagcctgaacggctttccagaaggagtgaggatgagcagagatgggggaaaggacctggccaagacagagggaagaaggggagccaggacagcggggctccgggggaggccatggagagactgggaagagcgcagaggtgtgacgacagtccagcacccagaaaagagcgactggcaaacaaggaccggccagccttgcagctgtatgatccaggagctcgcttccgagcgcgagagtgtggcggaaacaggaggatctgcaaggcagaaggttcggggactggtcctgagaagagggaagaggcagagtgagtcactgcacgcacctggcctccatggacgagcaagggcatcccagaaacgtgtaaatgaccccgagtgtgactgggaaggagaacttattccttaccaggaaactggaagctaaaaatacagagggtgacgtagaaacacgcagaaaccattctaaagaaagtagtgatcttgtattaaattgagcagaattctcacagattttaccattcctgttataaactagtatttgttgtttagccaaaacagaaaatgatttccactggacagtagaaaaatatgtgtaaaatagggaagaaagttagtattggatcagtgtgagtcctgaagcactttcagtgctgtgagaacgacatccactttgggtttcattcgtttgtaagcagaggagctgtcagtcactcgtgcttctcggtggcctctgagccatggtgtcgagtgaagagtagttcttgtttgttacaacctttgtgagtcagccatgcccgcaaagcgtgctgtgttttagtcctggtaggaatatttatcagagttcacactatataaaacccaacagcttcaactattgccctttcaacagttttgccactgaccggatagaaacggtttcagtctctggatggatgtgtttgtggtttgtaaccattacggtttaaaccatggtttaagaatttgcccaaataacagaaattttgttcgggaagggataaactagatatagcatacagagcctgtttttgagttttagatactttatttgtaaataacttaaaatagctttctgaaaccgtgcattctgtagtttcttcctttcagtgaaattgctaaatgtcaatgtatttttggcactgcgattttaaccatttattaaataaaaattttgttaaagaaaaaaaaaaaaaaa

SEQ ID NO: 19 - DNA, 1267 bp, Homo sapiens
  misc_feature: Incyte clone 843183
tgaccacgcgtccggccagagtgatcaatttttcctgggcctggagggccattacctacatctggctctactcactggcgtgggcaggagcacctctcctgggatggaacaggtacatcctggacgtacacggactaggctgcactgtggactggaaatccaaggatgccaacgattcctcctttgtgcttttcttatttcttggctgcctggtggtgcccctgggtgtcatagcccattgctatggccatattctatattccattcgaatgagagaggtgactcataacaacgatggagacatagctacacacttgagaatgaagattcacagtgaaattctttgaaaatctacagatgtcttggattaaggagcactaggcaccagccatttatacagccagagaacaatctgatagagaattctgaagaatttgttttagctggttcattctttaggacaaatttgtgcctgtgccaatcatttcaaaattgcttcatccatagcaattttgaaaactatcctcaaatgattgaataaagaggcttcgttgtgtggaagatcttcagacaattcaagtgatcaagattttaaaatatgaaaagaaactggccaaaatgtgctttttaatgatattcaccttcctggtctgttggatgccttatatcgtgatctgcttcttggtggttaatggtcatggtcacctggtcactccaacaatatctattgtttcgtacctctttgctaaatcgaacactgtatacaatccagtgatttatgtcttcatgatcagaaagtttcgaagatcccttttgcagcttctgtgcctccgactgctgaggtgccagaggcctgctaaagacctaccagcagctggaagtgaaatgcagatcagacccattgtgatgtcacagaaagatggggacaggccaaagaaaaaagtgactttcaactcttcttccatcatttttatcatcaccagtgatgaatcactgtcagttgacgacagcgacaaaaccaatgggtccaaagttgatgtaatccaagttcgtcctttgtaggaatgaagaatggcaacgaaagatggggccttaaattggatgccacttttggactttcatcataagaagtgtctggaatacccgttctatgtaatatcaacagaaccttgtggtccagcaggaaatccgaattgcccatatgctcttgggcctcaggaagaggttgaacaaaaacaaattcttttaattcaacgggtgctttaca

SEQ ID NO: 20 - DNA, 1691 bp, Homo sapiens
  misc_feature: Incyte clone 965938
ccgtagaccgtcatctgatttggtcttttgatttgggtgtactcttccagcatgtgtcttaaagcttacataagtggtatttgcctcttttccactaagcaatttttaaaaaaattcatcccaactttcatcacatcatcttatgtggaagtgccgtagtccataccagttccctgatttaggacacagaggttgtttctgattttcagctaacaggaattatgccgtcaaactccttgtaagagtcttttccaacatgcacgtgcatctcaagagtgggggtgacttgtagaagcggagggaagagctcagtggttttgccttcgactcctgactccctgcgtgcccctcattgccctgacccaactggacctgaccctgtgtccccctcccacagtccacgctggcagtgtgggtgaagtggcccatccatcccagcgccccagagcttgcgggccacaagagacagtttggctctgtctgccaccaggatcccagggtgtgtgatgagccctcctccgaagaccctcatgagtggccagaagacatcaccaagtggccgatctgcaccaaaaacagcgctgggaaccacaccaaccatccccacatggactgtgtcatcacaggacggccctgctgcattggcaccaagggcaggtgtgagatcacctcccgggagtactgtgacttcatgaggggctacttccatgaggaggccacgctctgctctcaggtgcactgcatggatgatgtgtgtgggctcctgccttttctcaaccccgaggtgcctgaccagttctaccgcctgtggctatccctcttcctgcacgccgggatcttgcactgcctggtgtccatctgcttccagatgactgtcctgcgggacctggagaagctggcaggctggcaccgcatagccatcatctacctgctgagtggtgtcaccggcaacctggccagtgccatcttcctgccataccgagcagaggtgggtcctgctggctcccagttcggcatcctggcctgcctcttcgtggagctcttccagagctgggcagatcctgggcgcggccctggcgtgccttcttcaagctgctggctgtggtgctcttcctcttcacctttgggctgctgccgtggattgacaactttgcccacatctcggggttcatcagtggcctcttcctctccttcgccttcttgccctacatcagctttggcaagttcgacctgtaccggaaacgctgccagatcatcatctttcaggtggtcttcctgggcctcctggctggcctggtggtcctcttctacgtctatcctgtccgctgtgagtggtgtgagttcctcacctgcatccccttcactgacaagttctgtgagaagtacgaactggacgctcagctccactgagctggctgcgggctccagcggccgtgtgctccagcaggccagagccagacacgacctccctgagcctcacaggcttacaggagtcacctgctccatgtggggactggcctgtttcctgaacacagacctctttcttgtgccttgttcacttctgttgaacccctcgtactgccgggcatttattatactacttcctgtcataaccttctaacttgtttcttgacgaccacctcatgtggccaataaatggactgggagcgttttagctgccattaacttga

SEQ ID NO: 21 - DNA, 1401 bp, Homo sapiens
  misc_feature: Incyte clone 1441620
gggtgacctctggggtgaggaaactgcgactgggagcgggacccaggcgtgcagcattcgccatgctccgctcacgcgtgggagactgggctgtggggtaccggcccggaaagcacgcagcctccaaagccgccttcctcagggaaatttgcgtgaccttactgccctccgtctacaggccttgtacctctccaggccgatttttccacaatttaaatcccagttcacctggtatccagctccagcaacttagagcgtttcacgtcacgccgggcgccaggcgtcggcttgtataacctgaaaacgctcctgtttttctcatctgtgcagtgggttttgattcccaccatggccatcacccagtttcggttatttaaattttgtacctgcctagcaacagtattctcattcctaaagagattaatatgcagatctggcagaggacggaaattaagtggagaccaaataactttgccaactacagttgattattcatcagttcctaagcagacagatgttgaagagtggacttcctgggatgaagatgcacccaccagtgtaaagatcgaaggagggaatgggaatgtggcaacacaacaaaattctttggaacaactggaacctgactattttaaggacatgacaccaactattaggaaaactcagaaaattgttattaagaagagagaaccattgaattttggcatcccagatgggagcacaggtttctctagtagattagcagctacacaagatctgccttttattcatcagtcttctgaattaggtgacttagatacctggcaggaaaataccaatgcatgggaagaagaagaagatgcagcctggcaagcagaagaagttctgagacagcagaaactagcagacagagaaaagagagcagccgaacaacaaaggaagaaaatggaaaaggaagcacaacggctaatgaagaaggaacaaaacaaaattggtgtgaaactttcataacacatgttcaaattttatcatgccagtaggagaaatctcagctccacaacccaagcaacatttgtatggatttaagagtattttaagaagacatactgcttgattttaatacattgatcaggccatccaggacaccacgattctcccaaagtaccttgaactcttagtgattgagactcaaaaaaacaaaaaagacttgagacaatgttttcttcaacatgctccaaatataagacatttgtttgctgtacagaaagtatcacaaatggaatatatcagtacctctcaagctagtgtttctagctaaataaatgggtgtatataattttatggtggaaaagaactgtactgtctgttatgatttccttcaatgtgcataatgataaaatagggaatgctacaaggccgggt

SEQ ID NO: 22 - DNA, 1987 bp, Homo sapiens
  misc_feature: Incyte clone 1510911
gggcgctgcaggtgtcggggcctcaaccttgcggaccgacagccatcgatcctcgggtggcctcgaggtggtggcagggccgccccctgcagtccggagacgaacgcacggaccgggcctccggaggcaggttcggctggaaggaaccgctctcgcttcgtcctacacttgcgcaaatgtctccgagcttactcacatagcatattggtatatcaaaatgaaatgcaaggaaccaaaaataacataattgaaggcagtaaaagtgaaattaaataggaagatcatcagtcaaggaagacccactggagaggacagaaaatgaagcagtgttttatcatgtgtatttcagcaggtcttcttgaaatttaactaaaaatatgactgctctctcttcagagaactgctcttttcagtaccagttacgtcaaacaaaccagcccctagatgttaactatctgctattcttgatcatacttgggaaaatattattaaatatccttacactaggaatgagaagaaaaaacacctgtcaaaattttatggaatatttttgcatttcactagcattcgttgatcttttacttttggtaaacatttccattatattgtatttcagggattttgtacttttaagcattaggttcactaaataccacatctgcctatttactcaaattatttcctttacttatggctttttgcattatccagttttcctgacagcttgtatagattattgcctgaatttctctaaaacaaccaagctttcatttaagtgtcaaaaattattttatttctttacagtaattttaatttggatttcagtccttgcttatgttttgggagacccagccatctaccaaagcctgaaggcacagaatgcttattctcgtcactgtcctttctatgtcagcattcagagttactggctgtcatttttcatggtgatgattttatttgtagctttcataacctgttgggaagaagttactactttggtacaggctatcaggataacttcctatatgaatgaaactatcttatattttcctttttcatcccactccagttatactgtgagatctaaaaaaatattcttatccaagctcattgtctgttttctcagtacctggttaccatttgtactacttcaggtaatcattgttttacttaaagttcagattccagcatatattgagatgaatattccctggttatactttgtcaatagttttctcattgctacagtgtattggtttaattgtcacaagcttaatttaaaagacattggattacctttggatccatttgtcaactggaagtgctgcttcattccacttacaattcctaatcttgagcaaattgaaaagcctatatcaataatgatttgttaatattattaattaaaagttacagctgtcataagatcataattttatgaacagaaagaactcaggacatattaaaaaataaactgaactaaaacaacttttgccccctgactgatagcatttcagaatgtgtcttttgaagggctatgataccatttattaaatagtgttttattttaaaaacaaaataattccaagaagtttttatagttattcagggacactatattacaaatattactttgttattaacacaaaaagtgataagagttaacatttggctatactgatgtttgtgttactcaaaaaaactactggatgcaaactgttatgtaaatctgagatttcactgacaactttaagatatcaacctaaacatttttattaaatgttcaaatgaaagcaagaaagtaaaaattggtcctaaaatgatttggcataaagttcaatgtaagagggaggaggaggattaccctccattttaaatggaggcaattaattttaaaaaggtataactcctggttttaatgccaataattcctgggaagagaaggggtttgggtggcccaccttatgggaactcgggaccgttgaca

SEQ ID NO: 23 - DNA, 1208 bp, Homo sapiens
  misc_feature: Incyte clone 2022379
cctaaatcccgacagctttatagagcccaggcctggcaggctcccagaacttgaagccaccagaccccacatggaaccaaaggcctcctgtccagcagctgcacccttgatggagagaaaattccatgttcttgtgggtgtcacggggagtgtcgcagccctgaagttgcctcttctggtgtcaaagcttttggacattcctgggctggaagtagcagtggtcacaactgagagagccaaacatttctacagcccccaggacattcctgtcaccctctacagcgacgctgatgaatgggagatgtggaagagccgctctgacccagttctgcacattgacctgcggaggtgggcagacctcctgctggtggctcctcttgatgccaacactctggggaaggtggccagtggcatctgtgacaacttgcttacctgcgtcatgcgggcctgggaccgcagcaagcccctgctcttctgcccggccatgaacaccgccatgtgggagcacccgatcacagcgcagcaggtagaccagctcaaggcctttggctatgtcgagatcccctgtgtggccaagaagctggtgtgcggagatgaaggtctcggggccatggctgaagtggggaccatcgtggacaaagtgaaagaacgtcctcttccagcacagtggcttccagcagagttgacctgggatttctgtcatgggtgtccctctgtactcagaatgggttcaggccaagtcggtgaagatggatgttggcaaaataggaggataccctcatttgctgaatgggggacctgctctgagcctgcccaggggccaggcctgctccaggttaaactggacggaaggcccaggtctcagtttctttcaaccaggagaggccgctgcctagagcccctccccaccttttcctggatgggtgaggcaagccaggagagcaagcagtgttgtcctcacgggaggaggactgagcgactgggaaaactcggctctacatctcacccagaacggcttttagaaacaccacagctggagagtcctggctgagccttgggagtttcagctctttggcggggtgcccaggtgccatgcgatcagcgaagcctgcgagttggcaggactctgaggtttcctgcagaccatgccatgagattgaaggtgcggggaaataaagaaaaatcaccatttagaaaaaaaaaaaaaa

SEQ ID NO: 24 - DNA, 2030 bp, Homo sapiens
  misc_feature: Incyte clone 2024312
cagagaaggagtagcgcgttcgtgcgtcctagttccagtacagcgtggagggtttaggcagcgtgttctgattctttgcgggacggcgagcgcatttgtgctttgcccgccgcggcctaggaggccttttgaggccgcgtagtcggtgtttttgaactgactctacagcttctggcaggccgtgcggcgccctgacccggcctcaccatgttggtgctgtttgaaacgtctgtgggttacgccatctttaaggttctaaatgagaagaaacttcaagaggttgatagtttatggaaagaatttgaaactccagagaaagcaaacaaaatagtaaagctaaaacattttgagaaatttcaggatacagcagaagcattagcagcattcacagctctgatggagggcaaaatcaataagcagctgaaaaaagttctgaagaaaatagtaaaagaagcccatgaaccgctggcagtagctgatgctaaactaggaggggtcataaaggaaaagctgaatctcagttgtatccatagtcctgttgttaatgaacttatgagaggaattcgttcacaaatggatggattaatccctggggtagaaccacgtgaaatggcagctatgtgtcttggattggctcacagcctgtctcgatatagattgaagtttagcgctgataaagtagacacaatgattgttcaggcaatttccttgttagatgacttggataaagaactaaacaactacattatgcgatgtagagaatggtatggctggcatttccctgaattaggaaaaattatttcagataatttaacatactgcaagtgtttacagaaagttggcgataggaagaactatgcctctgccaagctttctgagttgctgccagaagaagttgaagcagaagtgaaagcagctgcagagatatcaatgggaacagaggtttcagaagaagatatttgcaatattctgcatctttgcacccaggtgattgaaatctctgaatatcgaacccagctctatgaatatctacaaaatcgaatgatggccattgcacccaatgttacagtcatggttggggaattagttggagcacggcttattgctcatgcaggttctcttttaaatttggccaagcatgcagcttctaccgttcagattcttggagctgaaaaggcacttttcagagccctcaaatctagacgggatacccctaagtatggtctcatttatcatgcttcactcgtgggccagacaagtcccaaacacaaaggaaagatttctcgaatgctggcagccaaaaccgttttggctatccgttatgatgcttttggtgaggattcaagttctgcaatgggagttgagaacagagccaaattagaggccaggttgagaactttggaagacagagggataagaaaaataagtggaacaggaaaagcattagcaaaaacagaaaaatatgaacacaaaagtgaagtgaagacttacgatccttctggtgactccacacttccaacctgttctaaaaaacgcaaaatagaacaggtagataaagaggatgaaattactgaaaagaaagccaaaaaagccaagattaaagttaaagttgaagaagaggaagaagaaaaagtggcagaagaagaagaaacatctgtgaagaagaagaagaaaaggggtaaaaagaaacacattaaggaagaaccactttctgaggaagaaccatgtaccagcacagcaattgctagtccagagaaaaagaagaaaaagaaaaaaaagagagagaacgaggattaacagaaaggaattacgattatatcacccggacacacatcatgcttaagattcaactgggagcataccagggatgctctctaacgtaatcaagggaaggttcagtaagacaaagtgatttatcatctataacttcaaacctatttgtcttgacatcaactctgttaaccttatgtcatcatttcttagagtctttgatatacaaataaaattttctttgtattttaaaaaaaaaa

SEQ ID NO: 25 - DNA, 1919 bp, Homo sapiens
  misc_feature: Incyte clone 2057886
gtccgggtccgctgcctggcgctgcgggcggcgggccatggtggtttggattgaaccgggcccggccggagcgccgagtcggagggggtggcagtgagcggcggcagaggctacggggctcggtttggctgactggggagtcggcaggcggcaggtcttttgtggggatgagccagggtgcaaggagagtacaatactccagttaccgaattgaaaccatccctgcagtggagcagcctcctccagtttctgttgggttttgagctacctgttaaataagtcagtgggattgtcaaggacaaagccctccctggctgcctcagggcaaaatcaggaaccatgcgaggccagcggagcctgctgctgggcccggcccgcctctgcctccgcctccttctgctgctgggttacaggcgccgctgtccacctctactccggggtctagtacagcgctggcgctacggcaaggtctgcctgcgctccctgctctacaactcctttgggggcagtgacaccgctgttgatgctgcctttgagcctgtctactggctggtagacaacgtgatccgctggtttggagtggtgttcgtggtcctggtgatcgtgctgacaggctccattgtagctatcgcctacctgtgtgtcctgcctctcatcctccgaacctactcagtgccacgactctgctggcatttcttctatagccactggaatctgatcctgattgtcttccactactaccaggccatcaccactccgcctgggtacccaccccagggcaggaatgatatcgccaccgtctccatctgtaagaagtgcatttaccccaagccagcccgaacacaccactgcagcatctgcaacaggtgtgtgctgaagatggatcaccactgcccctggctaaacaattgtgtgggccactataaccatcggtacttcttctctttctgctttttcatgactctgggctgtgtctactgcagctatggaagttgggaccttttccgggaggcttatgctgccattgagacttatcaccagaccccaccacccaccttctcctttcgagaaaggatgactcacaagagtcttgtctacctctggttcctgtgcagttctgtggcacttgccctgggtgccctaactgtatggcatgctgttctcatcagtcgaggtgagactagcatcgaaaggcacatcaacaagaaggagagacgtcggctacaggccaagggcagagtatttaggaatccttacaactacggctgcttggacaactggaaggtattcctgggtgtggatacaggaaggcactggcttactcgggtgctcttaccttctagtcacttgccccatgggaatggaatgagctgggagccccctccctgggtgactgctcactcagcctctgtgatggcagtgtgagctggactgtgtcagccacgactcgagcactcattctgctccctatgttatttcaagggcctccaagggcagcttttctcagaatccttgatcaaaaagagccagtgggcctgccttagggtaccatgcaggacaattcaaggaccagcctttttaccactgcagaagaaagacacaatgtggagaaatcttaggactgacatccctttactcaggcaaacagaagttccaaccccagactaggggtcaggcagctagctacctaccttgcccagtgctgacccggacctcctccaggatacagcactggagttggccaccacctcttctacttgctgtctgaaaaaacacctgactagtacagctgagatcttggcttctcaacagggcaaagataccaggcctgctgctgaggtcactgccacttctcacatgctgcttaagggagcacaaataaaggtattcgatttttaaaaaaaaaaaaaaaaaaaaaa

SEQ ID NO: 26 - DNA, 1943 bp, Homo sapiens
  misc_feature: Incyte clone 2121924
aggagcgaggagcagctcgggagagccggagcggtagcagcagcagcggcggcggcggcggcggcgaggctcggcgccctcttccctgcaaaccatgtttgccaaaggcaaaggctcggcggtgccctcggatgggcaggctcgggaaaagttagctttatacgtctacgaatatttactgcacgtaggagcacagaaatctgcacagaccttcttatcggagattcgatgggaaaaaaacatcacgttgggagaaccgcctgggtttttgcactcgtggtggtgtgtattttgggacctttactgtgcagctcctgaaaggagagacacttgtgaacattcaagtgaagcaaaagcctttcatgattatagtgcagcagctgccccgagccccgtgcttggcaacattccccccaacgatgggatgccgggaggccccatcccgccaggtttctttcagccttttatgtcaccgcgatacgcaggcggccccaggcccccgatcagaatgggaaaccagcctccgggaggagttcctgggacacagccattgctgcccaattctatggatcccacacgacaacaaggccaccccaacatgggaggatcaatgcagagaatgaaccctccccgaggcatggggcccatgggtcccggcccacagaattacggcagtggcatgagaccaccacccaactccctcggccccgccatgcccgggattaacatgggcccgggagctggcagaccctggcccaatcctaacagtgctaactcaattccatactcctcctcatcacctggtacctatgtgggaccccctggtggtggcggtcctccaggaacacccattatgcccagtcccgcagattcaacaaattccagtgacaacatctacacaatgattaatccagtgccgcctggaggcagccggtccaacttcccgatgggtcccggctcggacggtccgatgggcggcatgggtggcatggagccacaccacatgaatggatcattagggtcaggcgacatagacggacttccaaaaaattctcctaacaacataagtggcattagcaatcctccaggcacccctcgagatgacggcgagctaggagggaacttcctccactcctttcagaacgacaattattctccaagcatgacgatgagtgtgtgatccccccttctccgagacgctgagagagcaggcattgcaggcgggaagatgccagaaattatgcaagaagtgaggtgtcattatccaggagctggtggggagggcatctccctgctcccctcaaccccctcccaccccatccacgccccctacctttcccaattttagtttcatgcaataaaaaggccaaactttttattccataaaacaagaaggacaaaactctcaaaaatgtatttcaagtcagtgaccagaaaaatcccaccccttgccctttccccaaaggaccttttctgtacatgacacttttttgttgttttttgtttggggttttaccattgttgggatttttttatttgttttcaggggggttttttgggggaaaatttttttaaatggaagcttctagcaagccccccaccccaatcaacctctatgctttcttcttaaaaaaaaaaaaaaagggaaaaggaaaaaaaaaaaagggaaaaccagaagccctgctgtctgtctggggcccaagccctttcaccagaaaagctagtctaggtgtgagagcccacattgtctgtagccatcaaaaataataataataaactgggacagtttaccaatccaaaaaaaaaaaaaaaaaaggggcgggccgcttctagaaggattcaaagcttaccgtaacgcggtgcaatgcgacggtccaaagccccttcttaaaggggtcccctaaaattccaaattcacctggcccgtcggtttttacaaccgg

SEQ ID NO: 27 - DNA, 1174 bp, Homo sapiens
  misc_feature: Incyte clone 2122815
ggcaacgcagctcgcggcgggcgctgggcgcgggatccgactctagtcgtaatggaggcgggcggctttctggactcgctcatttacggagcatgcgtggtcttcacccttggcatgttctccgccggcctctcggacctcaggcacatgcgaatgacccggagtgtggacaacgtccagttcctgccctttctcaccacggaagtcaacgtgttgtgctcctacagactgcaaccctgctaggggtccttctcctgggttatggctacttttggctcctggtacccaaccctgaggcccggcttcagcagttgggcctcttctgcagtgtcttcaccatcagcatgtacctctcaccactggctgacttggctaaggtgattcaaactaaatcaacccaatgtctctcctacccactcaccattgctacccttctcacctctgcctcctggtgcctctatgggtttcgactcagagatccctatatcatggtgtccaactttccaggaatcgtcaccagctttatccgcttctggcttttctggaagtaccccaggagcaagacaggaactactggctcctgcaaacctgaggctgctcatctgaccactgggcaccttagtgccaacctgaaccaaagagacctccttgtttcagctgggcctgctgtccagcttcccaggtgcagtgggttgtgggaacaagagatgactttgaggataaaaggaccaaagaaaaagctttacttagatgattgattggggcctaggagatgaaatcactttttattttttagagatttttttttttaattttggaggttggggtgcaatctttagaatatgccttaaaaggccgggcgcggtggctcacgcctgtaatcccagcactttgggaggccaaggtgggcggatcgcctgaggtcaggagttcaagaccaacctgactaacatggtgaaaccccatctctactaaaaatacaaaattagccaggcatgatggcacatgcctgtaatcccagatacttgggaggctgaggcaggagaattgcttgaacccaggaggtggaggttgcagtgagctgagatcgtgccattgtgatatgaatatgccttatatgctgatatgaatatgccttaaaataaagtgttccccacccctgccaaaaaaaaaa

SEQ ID NO: 28 - DNA, 1374 bp, Homo sapiens
  misc_feature: Incyte clone 2132179
cggtccaggcctctggcgaacatggcgcttgtcccctgccaggtgctgcggatggcaatcctgctgtcctactgctctatcctgtgtaactacaaggccatcgaaatgccctcacaccagacctacggagggagctggaaattcctgacgttcattgatctggttatccaggctgtcttttttggcatctgtgtgctgactgatctttccagtcttctgactcgaggaagtgggaaccaggagcaagagaggcagctcaagaagctcatctctctccgggactggatgttagctgtgttggcctttcctgttggggtttttgttgtagcagtgttctggatcatttatgcctatgacagagagatgatatacccgaagctgctggataattttatcccagggtggctgaatcacggaatgcacacgacggttctgccctttatattaatcgagatgaggacatcgcaccatcagtatcccagcaggagcagcggacttaccgccatatgtaccttctctgttggctatatattatgggtgtgctgggtgcatcatgtaactggcatgtgggtgtaccctttcctggaacacattggcccaggagccagaatcatcttctttgggtctacaaccatcttaatgaacttcctgtacctgctgggagaagttctgaacaactatatctgggatacacagaaaagtatggaagaagagaaagaaaagcctaaattggaatgagatccaagtctaaacgcaagagctagattgagccgccattgaagactccttcccctcgggcattggcagtgggggagaaaaggcttcaaaggaacttggtggcatcagcacccccctcccccaatgaggacaccttttatatataaatatgtataaacatagaatacagttgtttccaaaagaactcaccctcactgtgtgttaaagaattcttcccaaagtcattactgataataacattttttccttttctagttttaaaaccagaattggaccttggatttttattttggcaattgtaactccatctaatcaagaaagaataaaagtttattgcacttctttttgagaaatatgttaaagtcaaaggggcatatatagagtaaggcttttgtgtatttaatcctaaaggtggctgtaatcatgaacctaggccaccatggggacctgagagggaaggggacagatgtttctcattgcataatgtcacagttgcctcaaatgagcaccatttgtaataatgatgtcaatttcatgaaaagcctgagtgtattgcatctcttgatttaatcatgtgaaacttttcctagatgcaaatgctgactaataaagacaaagccaccctgaaaaaaaaaa

SEQ ID NO: 29 - DNA, 1498 bp, Homo sapiens
  misc_feature: Incyte clone 2326441
cacagctgtgatcagtccccaggatcccacgcttctcatcggctcctccctgctggccacctgctcagtgcacggagacccaccaggagccaccgccgagggcctctactggaccctcaatgggcgccgcctgccccctgagctctcccgtgtactcaacgcctccaccttggctctggccctggccaacctcaatgggtccaggcagcggtcgggggacaacctcgtgtgccacgcccgtgacggcagcatcctggctggctcctgcctctatgttggcctgcccccagagaaacccgtcaacatcagctgctggtccaagaatcatgaaggacttgacctgccgctggacgccaggggcccacggggagaccttcctccacaccaactactccctcaagtacaagcttaggtggtatggccaggacaacacatgtgaggagtaccacacagtggggccccactcctgccacatccccaaggacctggctctctttacgccctatgagatctgggtggaggccaccaaccgcctgggctctgcccgctccgatgtactcacgctggatatcctggatgtggtgaccacggaccccccgcccgacgtgcacgtgagccgcgtcgggggcctggaggaccagctgagcgtgcgctgggtgtcgccacccgccctcaaggatttcctctttcaagccaaataccagatccgctaccgagtggaggacagtgtggactggaaggtggtggacgatgtgagcaaccagacctcctgccgcctggccggcctgaaacccggcaccgtgtacttcgtgcaagtgcgctgcaacccctttggcatctatggctccaagaaagccgggatctggagtgagtggagccaccccacagccgcctccactccccgcagtgagcgcccgggcccgggcggcggggcgtgcgaaccgcggggcggagagccgagctcggggccggtgcggcgcgagctcaagcagttcctgggctggctcaagaagcacgcgtactgctccaacctcagcttccgcctctacgaccagtggcgagcctggatgcagaagtcgcacaagacccgcaaccagcacaggacgaggggatcctgccctcgggcagacggggcacggcgagaggtcctgccagataagctgtaggggctcaggccaccctccctgccacgtggagacgcagaggccgaacccaaactggggccacctctgtaccctcacttcagggcacctgagccaccctcagcaggagctggggtggcccctgagctccaacggccataacagctctgactcccacgtgaggccacctttgggtgcaccccagtgggtgtgtgtgtgtgtgtgagggttggttgagttgcctagaacccctgccagggctgggggtgagaaggggagtcattactccccattacctagggcccctccaaaagagtccttttaaataaatgagctatttaggtgcaaaaaaaaaa

SEQ ID NO: 30 - DNA, 1440 bp, Homo sapiens
  unsure 9, 43, 58, 68, 1430-1440: a or g or c or t, unknown, or other
cttgtggantaaccgttattacccgccttttgattgagcttantccccgtttgccggnagccggacgnattcggcacaggcgtccgctgcagtccgccggcgagggagttacgcacgtcctgattctcctggagtctccagcccgcccagtggccgcagtcacccaggtccagaggcggcggtatcacaggctctccgacatgtctatgctggctgaacgtcggcggaagcagaagtgggctgtggatcctcagaacactgcctggagtaatgacgattccaagtttggccagcggatgctagagaagatggggtggtctaaaggaaagggtttaggggctcaggagcaaggagccacagatcatattaaagttcaagtgaaaaataaccacctgggactcggagctaccatcaataatgaagacaactggattgcccatcaggatgattttaaccagcttctagccgaactgaacacttgccatgggcaggaaaccacagattcctcggacaagaaggaaaagaaatcttttagccttgaggaaaagtccaaaatctccaaaaaccgtgttcactatatgaaattcacaaaagggaaggatctgtcatctcggagcaaaacagatcttgactgcatttttgggaaaagacagagtaagaagactcccgagggcgatgccagtccctccactccagaggagaacgaaaccacgacaaccagcgccttcaccatccaggagtactttgccaagcggatggcagcactgaagaacaagccccaggttccagttccagggtctgacatttctgagacgcaggtggaacgtaaaagggggaagaaaagaaataaagaggccacaggtaaagatgtggaaagttacctccagcctaaggccaagaggcacacggagggaaagcccgagagggccgaggcccaggagcgagtggccaagaagaagagcgcgccagcagaagagcagctcagaggcccctgctgggaccagagttccaaggcctctgctcaggatgcaggggaccatgtgcagccgcctgagggccgggacttcaccctgaagcccaaaaagaggagagggaagaaaaagctgcaaaaaccagtagagatagcagaggacgctacactagaagaaacgctagtganaaaagaagaagaagaaagattccaaatgaatccttcccagccggggccttccgaccactcagctgtcagggcactgcgggggcagacacctctggcctgaagtcacagcagagttcaccccagagcgtctgggcgcatcttgtggcatgcccatgggctgccgagtcctgccctctcgccacatttcccccaagttacattcccaggaggacctttttaatgttctcaatcgtggctctcagacacaaataaatttttttgtaaactctgnnnnnnnnnnn

SEQ ID NO: 31 - DNA, 1251 bp, Homo sapiens
  misc_feature: Incyte clone 2936050
gcctcggtactgacctctgcagagccgggtggagcccattgacgtccagcgaagatcggagcagcgatggacggtcgggtgcagctgataaaggccctcctggccttgccgatccggcccgcggcgcgtcgctggaggaacccgattccctttcccgagacgtttgacggcgataccgaccgactcccggagttcatcgtgcagacgggctcctacatgttcgtggacgagaacacgttctccagcgacgccctgaaggtgacgttcctcatcacccgcctcacggggcccgccctgcagtgggtgatcccctacatcaagaaggagagccccctcctcaatgattaccggggctttctggccgagatgaagcgagtctttggatgggaggaggacgaggacttctaggccgggagaccctcgggcctgggggcgggtgctctgggaagagttcgctgtgttactggccaccgctagggtctccacaggcgccctccctcccccgcaccctccccctcgagccgccgcgatgtcccctgcgctcctgtcccctcccgcgtagtgcttgcctttgttccaggaatagcgctccaggctcctgctgcagctcctgggccgcactcttgagcgcgctgcctccgccctcttttgccagccccagcccctccatgcacatttggacgctgtcctgcacttcaggtgcaagctgggctcctgttacatactggacagaccacccacagccgctgctgccaacccacctccactcctccagactgccagccaactacatcattctgcccacagacctacgctaccactgccatcgccatccaccgcatctcaccaacagactgtggctcctagtgatgtggactcacctcggaggtatccgagctggacacagcccctggacagtgatccagacagctggccgtcccccaaggagcctgtcaccttcagcgagacccatttcctccccatccccagagacctcttgtgttcctgccacatagctgccagggcttaagtgtgcctggcaaccaaatcgaatctctcattttctcctgtggaccagttagttttgcctagaatcctgttttcttctgaatttgcagctgtctctctgatgggtgccttttgttcaacacagtaagccctgctcccttccctgctctaatacactacctgtacaaaggttttttccttatttttaataaatgtcagacactattaaatagaaaaaaaaaa

SEQ ID NO: 32 - DNA, 1211 bp, Homo sapiens
  misc_feature: Incyte clone 3428945
gatctggtaaagcccccatcctggtctgacactgtcatgggtaccagtctcctatgctgggtggtcctgggtttcctagggacagattctgtttccacagatcacacaggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgctgtgctagcagcttagcgacagggggaaccggggagctgttttttggagaaggctctaggctgaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatt

(19) United States
(12) Patent Application Publication
(10) Pub. No.: US 2005/0079575 A1
(43) Pub. Date: Apr. 14, 2005

(54) Human receptor molecules

(76) Inventors: Jennifer L. Hillman, Mountain View, CA (US); Olga Bandman, Mountain View, CA (US); Y. Tom Tang, San Jose, CA (US); Henry Yue, Sunnyvale, CA (US); Preeti Lal, Santa Clara, CA (US); Neil C. Corley, Mountain View, CA (US); Karl J. Guegler, Menlo Park, CA (US); Chandra Patterson, Mountain View, CA (US)

Correspondence Address: FOLEY AND LARDNER;SUITE 500, 3000 K STREET NW, WASHINGTON, DC, 20007, US

(21) Appl. No.: 10/871,385

(22) Filed: Jun. 21, 2004

Related U.S. Application Data
(63) Continuation of application No. 09/071,822, filed on May 1, 1998, now abandoned.

(51) Int. Cl. C07K 14/705; C07H 21/04

(52) U.S. Cl. 435/69.1; 530/350; 435/320.1; 435/325; 514/12; 536/23.5

(57) ABSTRACT

The invention provides human receptor molecules (REC) and polynucleotides which identify and encode REC. The invention also provides expression vectors, host cells, antibodies, agonists, and antagonists. The invention also provides methods for diagnosing, treating or preventing disorders associated with expression of REC.

### FIELD OF THE INVENTION

This invention relates to nucleic acid and amino acid sequences of human receptor molecules and to the use of these sequences in the diagnosis, treatment, and prevention of neoplastic, immunological, reproductive, gastrointestinal, nervous, smooth muscle, and musculoskeletal disorders.

### BACKGROUND OF THE INVENTION

The term receptor describes proteins that specifically recognize other molecules. The category is broad and includes proteins with a variety of functions. The bulk of the proteins termed receptors are cell surface proteins which when they bind extracellular ligands, produce cellular responses in the areas of growth, differentiation, endocytosis, and immune response. Other receptors facilitate the specific transport of proteins out of the endoplasmic reticulum and localize enzymes to a particular location in the cell. The term may also be applied to proteins which act as receptors for ligands with known or unknown chemical composition which interact with other cellular components. For example, the steroid hormone receptors bind to and regulate transcription of genomic DNA.

Regulation of cell proliferation, differentiation, and migration is important for the formation and function of tissues. Secreted regulatory proteins such as growth factors coordinately control these cellular processes and act as mediators in cell-cell signaling pathways. Growth factors are secreted from the cell, and they bind to specific cell-surface receptors on target cells. The bound receptors trigger intracellular signal transduction pathways which activate various downstream effectors that regulate gene expression, cell division, cell differentiation, cell motility, and other cellular processes.

Cell surface receptors are typically integral membrane proteins of the plasma membrane. These receptors recognize hormones such as catecholamines; peptide hormones; growth and differentiation factors; small peptide factors; galanin, somatostatin, and tachykinins; and circulatory system-borne signaling molecules. Cell surface receptors on immune system cells recognize antigens, antibodies, and major histocompatibility complex (MHC)-bound peptide. Other cell surface receptors bind ligands to be internalized by the cell. This receptor-mediated endocytosis functions in the uptake of low density lipoproteins (LDL), transferrin, glucose- or mannose-terminal glycoproteins, galactose-terminal glycoproteins, immunoglobulins, phosphovitellogenins, fibrin, proteinase-inhibitor complexes, plasminogen activators, and thrombospondin (Lodish, H. et al. (1995) Molecular Cell Biology, Scientific American Books, New York N.Y., p. 723; and Mikhailenko, I. et al. (1997) J. Biol. Chem. 272:6784-6791).

Signal transduction is one process by which cells respond to extracellular signals (hormones, neurotransmitter, growth and differentiation factors, etc.) through a cascade of biochemical reactions. The process begins with the binding of the signal molecule to a cell membrane receptor and ends with the activation of an intracellular target molecule. Such processes regulate many cell functions including cell proliferation, differentiation, gene transcription, and oncogenic transformation.

Many growth factor receptors, including epidermal growth factor, platelet-derived growth factor, and fibroblast growth factor, contain intrinsic protein kinase activities. When the polypeptide growth factor binds to the receptor, it triggers the autophosphorylation of a tyrosine residue on the receptor. It is believed that these phosphorylated sites are recognition sites for the binding of other cytoplasmic signaling proteins in the signaling pathway that eventually links the initial receptor activation at the cell surface to the activation of a specific intracellular target molecule. These signaling proteins contain a common domain referred to as a src homology 2 (SH2) domain. SH2 domains are found in a variety of signaling molecules and oncogenic proteins such as phospholipase C-y, Ras GTPase activating protein, and pp60^{c-src} (Lowenstein, E. J. et al. (1992) Cell 70:431-42).

Epidermal growth factor (EGF) is a mitogen that stimulates the proliferation of epithelial tissue. In addition, some EGF-related proteins act as inductive signals in the differentiation of embryonic tissue. Proteins belonging to the EGF family share a conserved, repeated motif of about 40 amino acids with a characteristic distribution of cysteine residues (Nicola, N. A. (1994) Guidebook to Cytokines and Their Receptors, Oxford University Press, New York, N.Y., pages 194-197). These EGF motifs are also found in numerous proteins outside the EGF family, particularly in extracellular proteins important for various aspects of cell-cell signaling and recognition.

Extracellular stimuli which induce early response genes include growth factors, phorbol esters, okadaic acid, protein synthesis inhibitors, toxins, and abrupt changes in temperature, pH, and oxygen. The stimulus activates cell surface receptors and membrane bound molecules which initiate signaling cascades that induce the transcription of early response genes. These early response genes include the genes for cytokines; fos, myc, jun, the edg-1 receptor, and nuclear receptors, all of which have roles in cellular proliferation and differentiation.

Many cell surface receptors have seven transmembrane regions, with an extracellular N-terminus that binds ligand and a cytoplasmic C-terminus that interacts with G proteins (Strosberg, A. D. (1991) Eur. J. Biochem. 196:1-10). Such G-protein coupled receptors (GPCRs) are integral membrane proteins characterized by the presence of such seven hydrophobic transmembrane domains which span the plasma membrane and form a bundle of antiparallel alpha helices. The transmembrane domains account for structural and functional features of the receptor. In most cases, the bundle of helices forms a binding pocket; however, when the binding site must accommodate more bulky molecules, the extracellular N-terminal segment or one or more of the three extracellular loops participate in binding and in subsequent induction of conformational change in intracellular portions of the receptor. The activated receptor, in turn, interacts with an intracellular heterotrimeric G-protein complex which mediates further intracellular signaling activities, generally interaction with guanine nucleotide binding (G) proteins and the production of second messengers such as cyclic AMP (cAMP), phospholipase C, inositol triphosphate or ion channel proteins (Baldwin, J. M. (1994) Curr. Opin. Cell Biol. 6:180-190).

The amino-terminus of the GPCR is extracellular, of variable length and often glycosylated; the carboxy-terminus is cytoplasmic and generally phosphorylated. Extracellular loops of the GPCR alternate with intracellular loops and link the transmembrane domains. The most conserved domains of GPCRs are the transmembrane domains and the first two cytoplasmic loops. GPCRs range in size from under 400 to over 1000 amino acids (Coughlin, S. R. (1994) Curr. Opin. Cell Biol. 6:191-197).

GPCRs respond to a diverse array of ligands including lipid analogs, amino acids and their derivatives, peptides, cytokines, and specialized stimuli such as light, taste, and odor. GPCRs function in physiological processes including vision (the rhodopsins), smell (the olfactory receptors), neurotransmission (muscarinic acetylcholine, dopamine, and adrenergic receptors), and hormonal response (luteinizing hormone and thyroid-stimulating hormone receptors).

GPCR mutations, which may cause loss of function or constitutive activation, have been associated with numerous human diseases (Coughlin, supra). For instance, retinitis pigmentosa may arise from mutations in the rhodopsin gene. Parma, J. et al. (1993, Nature 365:649-651) report that somatic activating mutations in the thyrotropin receptor cause hyperfunctioning thyroid adenomas and suggest that certain G-protein-coupled receptors susceptible to constitutive activation may behave as proto-oncogenes.

The frizzled cell surface receptor, originally identified in Drosophila melanogaster, is important for proper bristle and hair polarity on the wing, leg, thorax, abdomen, and eye of the developing insect (Wang, Y. et al. (1996) J. Biol. Chem. 271:4468-4476). The frizzled gene encodes a 587 amino acid protein which contains an N-terminal signal sequence and seven putative transmembrane regions. The cysteine-rich N-terminus is probably extracellular and the C-terminus is probably cytosolic. Multiple frizzled gene homologs have been found in rat, mouse, and human. The frizzled receptors are not homologous to other seven-transmembrane-region receptors and their ligands are still unknown.

T cells play a dual role in the immune system as effectors and regulators, coupling antigen recognition and the transmission of signals that induce cell death in infected cells and stimulate other immune cells. Although T cells recognize a wide range of different antigens, a particular clonal line of T cells can only recognize a single antigen and only when it is presented to the T cell receptor (TCR) as a peptide complexed with a major histocompatibility molecule (MHC) on the surface of antigen presenting cell. The TCR on most T cells consists of immunoglobulin-like integral membrane glycoproteins containing two polypeptide subunits, α and β, of similar molecular weight. The TCR β subunit has an extracellular domain containing both variable and constant regions, a transmembrane domain that traverses the membrane once, and a short intracellular domain (Saito, H. et al. (1984) Nature 309:757-762). The genes for the TCR subunits are constructed through somatic rearrangement of different gene segments. Interaction of antigen in the proper MHC context with the TCR initiates signaling cascades that induce the proliferation, maturation, and function of cellular components of the immune system (Weiss, A. (1991) Annu. Rev. Genet. 25: 487-510). Rearrangements in TCR genes and alterations in TCR expression have been noted in lymphomas, leukemias, autoimmune disorders, and immunodeficiency disorders (Aisenberg, A. C. et al. (1985) N. Engl. J. Med. 313:529-533; Weiss, supra; and Olive, supra).

Other potential membrane-spanning and membrane protein-interacting proteins with putative receptor function include the muf1 protein; MARCO; the transmembrane 4 family (TM4) of proteins; the dopamine, serotonin, and muscarinic receptors; and prenylated proteins.

Abnormal hormonal secretion is linked to disorders including diabetes insipidus, hyper- and hypoglycemia, Grave's disease and goiter, and Cushing's and Addison's diseases. Cancer cells secrete excessive amounts of hormones or other biologically active peptides. Disorders related to excessive secretion of biologically active peptides by tumor cells include fasting hypoglycemia due to increased insulin secretion from insulinoma-islet cell tumors; hypertension due to increased epinephrine and norepinephrine secreted from pheochromocytomas of the adrenal medulla and sympathetic paraganglia; and carcinoid syndrome, which includes abdominal cramps, diarrhea, and valvular heart disease, caused by excessive amounts of vasoactive substances secreted from intestinal tumors. Tumors may exhibit ectopic synthesis and secretion of biologically active peptides, including ACTH and vasopressin in lung and pancreatic cancers; parathyroid hormone in lung and bladder cancers; calcitonin in lung and breast cancers; and thyroid-stimulating hormone in medullary thyroid carcinoma.

Inflammation is a molecular, cellular, and tissue program during which foreign substances and pathogens are destroyed, and injured tissue is repaired through a variety of biochemical, biophysical, and cellular mechanisms. The principal cellular mediators of inflammation are leukocytes, particularly granulocytes and the monocytes/macrophages. Macrophages recognize, internalize, and destroy a variety of foreign (non-self) and endogenous substances and pathogens, including bacteria, parasites, and viruses. The exact recognition mechanism for non-self pathogens is unknown, but it has been proposed that receptors with broad binding specificity are used to discriminate between self and non-self antigens. Macrophages are also thought to play in important role in the immune response by presenting foreign antigens to lymphocytes.

Steroid hormones regulate many cellular and tissue functions. Progesterone, a 4-pregnene-3,20-dione derived from cholesterol, is a critical oscillating component of the female reproductive cycle. These oscillations correlate with anatomical and morphological changes including menstruation and pregnancy.

The activities of progesterone are mediated through the intracellular progesterone receptor (PR). In the cytoplasm PR associates with several other proteins and factors termed the PR heterocomplex (PRC). PR is inactive when bound by molecular chaperones, immunophillins, and the heat shock proteins (hsp70, hsp90, hsp27, and p59 (hsp56), p48 and p23; Johnson, J. L. et al. (1994) Mol. Cell. Biol. 14:1956-1963). Active PR binds progesterone and translocates to the nucleus where it binds as a transcription factor to canonical DNA transcriptional elements of progesterone-regulated genes implicated in differentiation and in the cell cycle (Moutsatsou, P and Sekeris, C. E. (1997) Ann. N.Y. Acad. Sci. 816:99-115).

Other non-membrane interacting receptor proteins include the small nuclear ribosomal proteins; the yeast growth-related SIS2 protein, single-stranded DNA-binding proteins, RAG-1 activating proteins, and the hamster FAR-17a protein.

The discovery of new human receptor molecules and the polynucleotides encoding them satisfies a need in the art by providing new compositions which are useful in the diagnosis, treatment, and prevention of neoplastic, immunological, reproductive, gastrointestinal, nervous, smooth muscle, and musculoskeletal disorders.

### SUMMARY OF THE INVENTION

The invention features substantially purified polypeptides, human receptor molecules, referred to collectively as “REC”. In one aspect, the invention provides a substantially purified polypeptide comprising an amino acid sequence selected from the group consisting of SEQ ID NOs:1-16, and fragments thereof.

The invention further provides a substantially purified variant having at least 90% amino acid identity to the amino acid sequences of SEQ ID NOs: 1-16, and fragments thereof. The invention also provides an isolated and purified polynucleotide encoding the polypeptide comprising an amino acid sequence selected from the group consisting of SEQ ID NOs:1-16, and fragments thereof. The invention also includes an isolated and purified polynucleotide variant having at least 90% polynucleotide sequence identity to the polynucleotide encoding the polypeptide comprising an amino acid sequence selected from the group consisting of SEQ ID NOs:1-16, and fragments thereof.

Additionally, the invention provides an isolated and purified polynucleotide which hybridizes under stringent conditions to the polynucleotide encoding the polypeptide comprising an amino acid sequence selected from the group consisting of SEQ ID NOs: 1-16, and fragments thereof, as well as an isolated and purified polynucleotide having a sequence which is complementary to the polynucleotide encoding the polypeptide comprising the amino acid sequence selected from the group consisting of SEQ ID NOs: 1-16, and fragments thereof.

The invention also provides an isolated and purified polynucleotide comprising a polynucleotide sequence selected from the group consisting of SEQ ID NOs: 17-32, and fragments thereof. The invention further provides an isolated and purified polynucleotide variant having at least 90% polynucleotide sequence identity to the polynucleotide sequence comprising a polynucleotide sequence selected from the group consisting of SEQ ID NOs: 17-32, and fragments thereof, as well as an isolated and purified polynucleotide having a sequence which is complementary to the polynucleotide comprising a polynucleotide sequence selected from the group consisting of SEQ ID NOs: 17-32, and fragments thereof.

The invention further provides an expression vector containing at least a fragment of the polynucleotide encoding the polypeptide comprising an amino acid sequence selected from the group consisting of SEQ ID NOs:1-16, and fragments thereof. In another aspect, the expression vector is contained within a host cell.

The invention also provides a method for producing a polypeptide comprising the amino acid sequence selected from the group consisting of SEQ ID NOs:1-16, and fragments thereof, the method comprising the steps of: (a) culturing the host cell containing an expression vector containing at least a fragment of a polynucleotide encoding the polypeptide under conditions suitable for the expression of the polypeptide; and (b) recovering the polypeptide from the host cell culture.

The invention also provides a pharmaceutical composition comprising a substantially purified polypeptide having the amino acid sequence selected from the group consisting of SEQ ID NOs: 1-16, and fragments thereof in conjunction with a suitable pharmaceutical carrier.

The invention further includes a purified antibody which binds to a polypeptide comprising the amino acid sequence selected from the group consisting of SEQ ID NOs: 1-16, and fragments thereof, as well as a purified agonist and a purified antagonist to the polypeptide.

The invention also provides a method for treating or preventing a neoplastic disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing an immunological disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing a reproductive disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing a gastrointestinal disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing a nervous disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing a smooth muscle disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for treating or preventing a musculoskeletal disorder, the method comprising administering to a subject in need of such treatment an effective amount of an antagonist of the polypeptide having an amino acid sequence selected from the group consisting of SEQ ID NOs:1-16, and fragments thereof.

The invention also provides a method for detecting a polynucleotide encoding the polypeptide comprising the amino acid sequence selected from the group consisting of SEQ ID NOs:1-16, and fragments thereof in a biological sample containing nucleic acids, the method comprising the steps of: (a) hybridizing the complement of the polynucleotide sequence encoding the polypeptide comprising the amino acid sequence selected from the group consisting of SEQ ID NOs:1-16, and fragments thereof to at least one of the nucleic acids of the biological sample, thereby forming a hybridization complex; and (b) detecting the hybridization complex, wherein the presence of the hybridization complex correlates with the presence of a polynucleotide encoding the polypeptide in the biological sample. In one aspect, the nucleic acids of the biological sample are amplified by the polymerase chain reaction prior to the hybridizing step.

### BRIEF DESCRIPTION OF THE TABLES

The first column of table 1 shows protein sequence identification numbers, SEQ ID NOs:1-16. The second column shows the nucleotide sequence identification numbers SEQ ID NOs:17-32 of the consensus sequences which encode SEQ ID NOs:1-16. The third column lists the Incyte Clone ID associated with the sequence identification number. The fourth column lists the tissue library from which the Incyte Clone was isolated. The fifth column lists the overlapping and/or extended nucleic acid sequences which were used to derive the consensus sequences SEQ ID NOs: 17-32.

The first column of table 2 lists the protein sequence identification numbers. The second column shows the number of amino acids of SEQ ID NOs: 1-16. The third column lists the potential phosphorylation sites available to cAMP- and cGMP-dependant protein kinases, casein kinase II, protein kinase C, and/or tyrosine kinases present in SEQ ID NOs:1-16. The fourth column lists the potential N-glycosylation sites present in SEQ ID NOs: 1-16. The fifth column lists any significant protein family signature or ligand/substrate binding motif present in SEQ ID NOs: 1-16. The sixth column names the GenBank database homolog with highest log-likelihood score of SEQ ID NOs: 1-16. The seventh column describes the method of analysis or algorithm(s) used to identify SEQ ID NOs:1-16.

The first column of table 3 lists the sequence identification number (SEQ ID NOs:1-16). The second column lists the tissue expression and fraction of tissue which express SEQ ID NOs:1-16. The third column lists the disease class and fraction of total diseases that express SEQ ID NOs:1-16. The fourth column lists the vector used to subclone the cDNA library.

### DESCRIPTION OF THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the cell lines, vectors, and methodologies which are reported in the publications and which might be used in connection with the invention. Nothing herein is to be construed as an admission that the invention is not entitled to antedate such disclosure by virtue of prior invention.

### Definitions

“REC” refers to the amino acid sequences of substantially purified REC obtained from any species, particularly a mammalian species, including bovine, ovine, porcine, murine, equine, and preferably the human species, from any source, whether natural, synthetic, semi-synthetic, or recombinant.

The term “agonist” refers to a molecule which, when bound to REC, increases or prolongs the duration of the effect of REC. Agonists may include proteins, nucleic acids, carbohydrates, or any other molecules which bind to and modulate the effect of REC.

An “allelic variant” refers to an alternative form of the gene encoding REC. Allelic variants may result from at least one mutation in the nucleic acid sequence and may result in altered mRNAs or in polypeptides whose structure or function may or may not be altered. Any given natural or recombinant gene may have none, one, or many allelic forms. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding REC include those sequences with deletions, insertions, or substitutions of different nucleotides, resulting in a polynucleotide the same as REC or a polypeptide with at least one functional characteristic of REC. Included within this definition are polymorphisms which may or may not be readily detectable using a particular oligonucleotide probe of the polynucleotide encoding REC, and improper or unexpected hybridization to allelic variants, with a locus other than the normal chromosomal locus for the polynucleotide sequence encoding REC. The encoded protein may also be “altered,” and may contain deletions, insertions, or substitutions of amino acid residues which produce a silent change and result in a functionally equivalent REC. Deliberate amino acid substitutions may be made on the basis of similarity in polarity, charge, solubility, hydrophobicity, hydrophilicity, and/or the amphipathic nature of the residues, as long as the biological or immunological activity of REC is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

The terms “amino acid” or “amino acid sequence” refer to an oligopeptide, peptide, polypeptide, or protein sequence, or a fragment of any of these, and to naturally occurring or synthetic molecules. In this context, “fragments,” “immunogenic fragments,” or “antigenic fragments” refer to fragments of REC which are preferably about 5 to about 20 amino acids in length, most preferably 15 amino acids, and which retain some biological activity or immunological activity of REC.

“Amplification” relates to the production of additional copies of a nucleic acid sequence. Amplification is generally carried out using polymerase chain reaction (PCR) technologies well known in the art.

“Antagonist” refers to a molecule which, when bound to REC, decreases the amount or the duration of the effect of the biological or immunological activity of REC. Antagonists may include proteins, nucleic acids, carbohydrates, antibodies, or any other molecules which decrease the effect of REC.

“Antibody” refers to intact molecules as well as to fragments thereof, such as Fab, F(ab′)₂, and Fv fragments, which are capable of binding the epitopic determinant. Antibodies that bind REC polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antigenic determinant” refers to that fragment of a molecule (i.e., an epitope) that makes contact with a particular antibody. When a protein or a fragment of a protein is used to immunize a host animal, numerous regions of the protein may induce the production of antibodies which bind specifically to antigenic determinants (given regions or three-dimensional structures on the protein). An antigenic determinant may compete with the intact antigen (i.e., the immunogen used to elicit the immune response) for binding to an antibody.

“Antisense” refers to any composition containing a nucleic acid sequence which is complementary to the “sense” strand of a specific nucleic acid sequence. Antisense molecules may be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

“Biologically active” refers to a protein having structural, regulatory, or biochemical functions of a naturally occurring molecule. Likewise, “immunologically active” refers to the capability of the natural, recombinant, or synthetic REC, or of any oligopeptide thereof, to induce a specific immune response in appropriate animals or cells and to bind with specific antibodies.

A “composition comprising a given polynucleotide sequence” or a “composition comprising a given amino acid sequence” refer broadly to any composition containing the given polynucleotide or amino acid sequence. The composition may comprise a dry formulation, an aqueous solution, or a sterile composition. Compositions comprising polynucleotide sequences encoding REC or fragments of REC may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

“Consensus sequence” refers to a nucleic acid sequence which has been resequenced to resolve uncalled bases, extended using XL-PCR (Perkin Elmer, Norwalk, Conn.) in the 5′ and/or the 3′ direction, and resequenced, or which has been assembled from the overlapping sequences of more than one Incyte Clone using a computer program for fragment assembly, such as the GELVIEW Fragment Assembly system (GCG, Madison, Wis.). Some sequences have been both extended and assembled to produce the consensus sequence.

The phrase “correlates with expression of a polynucleotide” indicates that the detection of the presence of nucleic acids, the same or related to a nucleic acid sequence encoding REC, by Northern analysis is indicative of the presence of nucleic acids encoding REC in a sample, and thereby correlates with expression of the transcript from the polynucleotide encoding REC.

A “deletion” refers to a change in the amino acid or nucleotide sequence that results in the absence of one or more amino acid residues or nucleotides.

“Derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

“Similarity” refers to a degree of complementarity. There may be partial similarity or complete similarity. The word “identity” may substitute for the word “similarity.” A partially complementary sequence that at least partially inhibits an identical sequence from hybridizing to a target nucleic acid is referred to as “substantially similar.” The inhibition of hybridization of the completely complementary sequence to the target sequence may be examined using a hybridization assay (Southern or Northern blot, solution hybridization, and the like) under conditions of reduced stringency. A substantially similar sequence or hybridization probe will compete for and inhibit the binding of a completely similar (identical) sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% similarity or identity). In the absence of non-specific binding, the substantially similar sequence or probe will not hybridize to the second non-complementary target sequence.

The phrases “percent identity” or “% identity” refer to the percentage of sequence similarity found in a comparison of two or more amino acid or nucleic acid sequences. Percent identity can be determined electronically, e.g., by using the MEGALIGN program (DNASTAR, Inc., Madison Wis.). The MEGALIGN program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.) Identity between sequences can also be determined by other methods known in the art, e.g., by varying hybridization conditions.

“Human artificial chromosomes” (HACs) are linear microchromosomes which may contain DNA sequences of about 6 kb to 10 Mb in size, and which contain all of the elements required for stable mitotic chromosome segregation and maintenance. (See, e.g., Harrington, J. J. et al. (1997) Nat Genet. 15:345-355.)

A “humanized antibody” refers to antibody molecules in which the amino acid sequence in the non-antigen binding regions has been altered so that the antibody more closely resembles a human antibody, and still retains its original binding ability.

“Hybridization” refers to any process by which a strand of nucleic acid binds with a complementary strand through base pairing.

“Hybridization complex” refers to a complex formed between two nucleic acid sequences by virtue of the formation of hydrogen bonds between complementary bases. A hybridization complex may be formed in solution (e.g., C₀t or R₀t analysis) or immobilized on an appropriate substrate.

“Insertion” or “addition” refer to changes in an amino acid or nucleotide sequence resulting in the addition of one or more amino acid residues or nucleotides, respectively, to the sequence found in the naturally occurring molecule.

“Immune response” can refer to conditions associated with inflammation, trauma, immune disorders, infectious or genetic disease, etc. These conditions can be characterized by expression of various factors, e.g., cytokines, chemokines, and other signaling molecules, which may affect cellular and systemic defense systems.

“Microarray” refers to an arrangement of distinct polynucleotides, i.e., array elements, on a substrate.

“Modulation” refers to a change in the activity of REC. For example, modulation may cause an increase or a decrease in protein activity, binding characteristics, or any other biological, functional, or immunological properties of REC.

“Nucleic acid sequence” refers to an oligomer, oligonucleotide, nucleotide or polynucleotide, and its fragments, and to DNA or RNA of genomic or synthetic origin which may be single- or double-stranded, and represent the sense or complementary (antisense) strand, a peptide nucleic acid (PNA), or a any DNA-like or RNA-like material. In this context, “fragments” refers to those nucleic acid sequences which are useful as probes or to produce an amino acid sequence which displays a useful biological or functional characteristic.

The terms “operably associated” or “operably linked” refer to functionally related nucleic acid sequences. A promoter is operably associated or operably linked with a coding sequence if the promoter controls the transcription of the nucleic acid sequence. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

“Oligonucleotide” refers to a nucleic acid sequence of at least about 6 nucleotides to 60 nucleotides, preferably about 15 to 30 nucleotides, and most preferably about 20 to 25 nucleotides, which can be used in PCR amplification, in hybridization, or on a microarray. “Oligonucleotide” is substantially equivalent to the terms “amplimer,” “primer,” “oligomer,” and “probe” as commonly defined in the art.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition which may also be pegylated to extend persistence in the cell. PNAs preferentially bind complementary single stranded DNA or RNA and stop replication, transcription or translation transcript elongation, which may be pegylated to extend their lifespan in the cell.

A biological “sample” refers to an extract from a cell, the cell, chromosomes isolated from a cell, genomic DNA, RNA, or cDNA in solution or bound to a substrate, REC, protein or fragments thereof, a bodily fluid, membrane isolated from a cell, etc.

“Specifically binding” refers to that interaction between a protein or peptide and an agonist, an antibody, or an antagonist. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

“Substantially purified” refers to nucleic acid or amino acid sequences that are removed from their natural environment and are isolated or separated, and are at least about 60% free, preferably about 75% free, and most preferably about 90% free from other components with which they are naturally associated.

A “substitution” refers to the replacement of one or more amino acids or nucleotides by different amino acids or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotides are bound.

“Transformation” describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

“Variant” refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE NAVIGATOR software.

### THE INVENTION

The invention is based on the discovery of new human receptor molecules, REC, the polynucleotides encoding REC, and the use of these compositions for the diagnosis, treatment, or prevention of neoplastic, immunological, reproductive, gastrointestinal, nervous, smooth muscle, and musculoskeletal disorders. Table 1 shows the protein and nucleotide SEQ ID NOs, Incyte Clone ID, library from which the cDNA was derived, and the overlapping and/or extended nucleic acid sequences, (identified by Incyte clone number and library) associated with the nucleic acid sequence for each of the human receptor molecules disclosed in the Sequence Listing.

As shown in table 2, each REC has been characterized with regard to its chemical and structural similarity with receptor molecules. In table 3, northern analysis shows the expression of this sequence in various libraries, at least 33% of which are immortalized or cancerous, at least 13% are in fetal tissue, and at least 13% of which involve immune response. Of particular note is the expression of REC in reproductive, gastrointestinal, nervous, smooth muscle, musculoskeletal, and endocrine tissues.

The invention also encompasses REC variants. A preferred REC variant is one which has at least about 80%, more preferably at least about 90%, and most preferably at least about 95% amino acid sequence identity to the REC amino acid sequence, and which contains at least one functional or structural characteristic of REC.

The invention also encompasses polynucleotides which encode REC. In a particular embodiment, the invention encompasses a polynucleotide sequence comprising the sequence selected from the group consisting of SEQ ID NOs:17-32, which encode REC.

The invention also encompasses a variant of a polynucleotide sequence encoding REC. In particular, such a variant polynucleotide sequence will have at least about 80%, more preferably at least about 90%, and most preferably at least about 95% polynucleotide sequence identity to the polynucleotide sequence encoding REC.

It will be appreciated by those skilled in the art that as a result of the degeneracy of the genetic code, a multitude of polynucleotide sequences encoding REC, some bearing minimal similarity to the polynucleotide sequences of any known and naturally occurring gene, may be produced. Thus, the

TABLE 1

| Protein | Nucleotide | Incyte Clone | | Fragment |
|---|---|---|---|---|
| SEQ ID NO | SEQ ID NO | ID | Library | Incyte Clone Number (Library) |
| 1 | 17 | 044150 | TBLYNOT01 | 044150 (TBLYNOT01), 2519387 (BRAITUT21), 1277252 (SCORNOT03), 1344825 |
| | | | | (PROSNOT11), 1299310 (BRSTNOT07), 2276961 (PROSNON01), 2055636 |
| | | | | (BEPINOT01), 1441890 (THYRNOT03) |
| 2 | 18 | 266775 | HNT2NOT01 | 266775 (HNT2NOT01), 2701310 (OVARTUT10), 2747851 (LUNGTUT11), 419110 |
| | | | | (BRSTNOT01), 1698964 (BLADTUT05), 1384229 (BRAITUT08), 2174644 |
| | | | | (ENDCNOT03), 1414813 (BRAINOT12), 216949 (STOMNOT01), 1381508 |
| | | | | (BRAITUT08) |
| 3 | 19 | 843183 | PROSTUT05 | 843183 (PROSTUT05) |
| 4 | 20 | 965938 | BRSTNOT05 | 965938 (BRSTNOT05), 2998414 (OVARTUT07), 1439653 (PANCNOT08), 2844144 |
| | | | | (DRGLNOT01), 1733210 (BRSTTUT08), 3498903 (PROSTUT13), 1856802 |
| | | | | (PROSNOT18) |
| 5 | 21 | 1441620 | THYRNOT03 | 1441620 (THYRNOT03), 2521640 (BRAITUT21), 1646155 (PROSTUT09), 723888 |
| | | | | (SYNOOAT01), 1987705 (LUNGAST01) |
| 6 | 22 | 1510911 | LUNGNOT14 | 1510911 (LUNGNOT14), 1488207 (UCMCL5T01), 3128735 (LUNGTUT12), 2472084 |
| | | | | (THP1NOT03), 2729646 (OVARTUT04), 2045427 (THP1T7T01), 1500290 |
| | | | | (SINTBST01) |
| 7 | 23 | 2022379 | CONNNOT01 | 2022379 (CONNNOT01), 3396623 (UTRSNOT16), 1376492 (LUNGNOT10) |
| 8 | 24 | 2024312 | KERANOT02 | 2024312 (KERANOT02), 3182120 (TLYJNOT01), 043915 (TBLYNOT01), 549794 |
| | | | | (BEPINOT01), 411761 (BRSTNOT01), 1638115 (UTRSNOT06) |
| 9 | 25 | 2057886 | BEPINOT01 | 2057886 (BEPINOT01), 2832301 (TLYMNOT03), 1521621 (BLADTUT04), 1823286 |
| | | | | (GBLATUT01), 1986582 (LUNGAST01) |
| 10 | 26 | 2121924 | BRSTNOT07 | 2121924 (BRSTNOT07), 2602788 (UTRSNOT10), 1319138 (BLADNOT04), 1750456 |
| | | | | (STOMTUT02), 1750456 (STOMTUT02), 1546334 (PROSTUT04), 1461157 |
| | | | | (PANCNOT04) |
| 11 | 27 | 2122815 | BRSTNOT07 | 2122815 (BRSTNOT07), 1698493 (BLADTUT05), 1211696 (BRSTNOT02), 334820 |
| | | | | (EOSIHET02) |
| 12 | 28 | 2132179 | OVARNOT03 | 2132179 (OVARNOT03), 2665629 (ADRENOT08), 085090 (LIVRNOT01), 667754 |
| | | | | (SCORNOT01) |
| 13 | 29 | 2326441 | OVARNOT02 | 2326441 (OVARNOT02), 1852256 (LUNGFET03) |
| 14 | 30 | 2825826 | ADRETUT06 | 2825826 (ADRETUT06), SBLA03401, SBLA01567, SBMA01690 |
| 15 | 31 | 2936050 | THYMFET02 | 2936050 (THYMFET02), 1676618 (BLADNOT05), 1267380 (BRAINOT09), 998794 |
| | | | | (KIDNTUT01), 1307910 (COLNFET02) |
| 16 | 32 | 3428945 | SKINNOT04 | 3428945 (SKINNOT04), 1909038 (CONNTUT01), 1451415 (PENITUT01) |

TABLE 2

| | Total | | | | | |
|---|---|---|---|---|---|---|
| | Amino | | Potential | | | |
| | Acid | | glycosylation | Signature | | |
| SEQ ID NO: | Residues | Potential Phosphorylation Sites | sites | Sequence | Top Hit | Method of Analysis |
| 1 | 610 | S448 T21 T26 S116 S261 S359 S426 T436 | N467 N499 | | muf1 gene product | BLAST |
| | | T469 T578 S145 S171 S254 S308 S352 | | | | |
| | | T501 | | | | |
| 2 | 275 | T14 T67 S128 S138 S140 S181 T266 T94 | | | hU1-70K protein | BLOCKS |
| | | S178 | | | | |
| 3 | 147 | T110 S98 T121 S127 S137 S131 | N112 N135 | | opsin | BLAST, BLOCKS, |
| | | | | | | PFAM |
| 4 | 336 | T21 S148 S265 T6 T21 T184 S218 S274 | N261 | | epidermal growth factor | BLAST, BLOCKS |
| | | | | | receptor-related protein | |
| 5 | 213 | S36 T44 T55 T61 S62 T151 S29 S70 T101 | | | cancer associated surface | PRINTS |
| | | T105 S128 | | | antigen | |
| 6 | 338 | T50 S86 T127 S131 S163 T199 T233 S236 | N8 N72 N123 | | D2 dopamine receptor | BLOCKS, PFAM, |
| | | | N218 | | | |
| 7 | 326 | S61 S71 S81 T186 S237 T243 S311 T53 | | | F25H9.6 | PRINTS |
| | | S268 T269 T301 | | | | |
| 8 | 529 | T335 S407 S121 S167 S247 S331 T397 | N103 N201 | | similar to S. cerevisiae | BLOCKS, PRINTS |
| | | S502 S219 S351 T409 T416 S440 T455 | N285 | | SIK1P | |
| | | S483 Y204 Y217 | | | | |
| 9 | 361 | T59 S241 S277 T281 T247 T322 | | | C43H6.7 gene product | BLOCKS, PFAM, |
| | | | | | | PRINTS |
| 10 | 361 | T79 T51 S256 T333 S126 | N49 N262 | | csdp | BLAST, PRINTS |
| | | | N305 N323 | | | |
| 11 | 221 | S38 S163 T207 | | | novel stromal cell protein | BLOCKS, PRINTS |
| 12 | 238 | T43 S84 S227 S38 T224 | | | androgen-dependent | BLAST, BLOCKS, |
| | | | | | expressed protein | PRINTS |
| 13 | 348 | T105 T127 S30 T74 S92 T136 S141 S176 | N90 N214 | G247-S253 | prolactin receptor hPRL | BLAST, BLOCKS, |
| | | S217 S243 T260 S263 S306 S320 | N304 | | receptor (alternatively | PFAM, PRINTS |
| | | | | | spliced) | |
| 14 | 352 | S152 T236 S23 S112 S139 T141 S163 T164 | N168 | | T23G7.3 | BLOCKS, PRINTS |
| | | T177 T205 T220 S273 T313 S103 S136 | | | | |
| | | S269 T290 | | | | |
| 15 | 210 | S20 S129 T155 S193 | | G16-S23 | putative prenylated | BLOCKS, PRINTS |
| | | | | | protein | |
| 16 | 318 | S316 T137 S156 S243 S31 T37 T44 S86 | N209 | | T-cell receptor beta | BLAST, BLOCKS, |
| | | T163 S216 | | | | PFAM, PRINTS |

TABLE 3

| SEQ ID NO: | Tissue Expression (Fraction of Total) | Disease Class (Fraction of Total) | Vector |
|---|---|---|---|
| 1 | Reproductive (0.309) Nervous (0.206) Cardiovascular (0.134) | Cancer (0.515) Fetal (0.196) Inflammation (0.134) | pBLUESCRIPT |
| 2 | Reproductive (0.281) Nervous (0.202) Gastrointestinal (0.169) | Cancer (0.506) Inflammation (0.180) Fetal (0.157) | pBLUESCRIPT |
| 3 | Gastrointestinal (0.188) Hematopoietic/Immune (0.188) | Cancer (0.375) Inflammation (0.375) Fetal (0.125) | pSPORT 1 |
| | Nervous (0.188) | | |
| 4 | Reproductive (0.432) Nervous (0.108) Cardiovascular (0.081) | Cancer (0.649) Inflammation (0.243) Fetal (0.216) | pSPORT 1 |
| 5 | Reproductive (0.289) Nervous (0.178) Cardiovascular (0.133) | Cancer (0.467) Inflammation (0.244) Trauma (0.133) | pINCY |
| 6 | Reproductive (0.267) Hematopoietic/Immune (0.233) | Cancer (0.567) Inflammation (0.300) Fetal (0.133) | pINCY |
| | Gastrointestinal (0.200) | | |
| 7 | Reproductive (0.333) Gastrointestinal (0.167) | Inflammation (0.417) Cancer (0.333) Fetal (0.167) | pINCY |
| | Hematopoietic/Immune (0.167) | | |
| 8 | Reproductive (0.229) Cardiovascular (0.157) | Cancer (0.457) Inflammation (0.229) Fetal (0.214) | pSPORT 1 |
| | Hematopoietic/Immune (0.157) | | |
| 9 | Cardiovascular (0.186) Reproductive (0.186) | Cancer (0.395) Inflammation (0.279) Fetal (0.256) | pSPORT 1 |
| | Hematopoietic/Immune (0.163) | | |
| 10 | Reproductive (0.286) Nervous (0.169) Gastrointestinal (0.117) | Cancer (0.481) Inflammation (0.195) Trauma (0.156) | pINCY |
| 11 | Reproductive (0.318) Gastrointestinal (0.182) | Cancer (0.477) Inflammation (0.341) Fetal (0.136) | pINCY |
| | Hematopoietic/Immune (0.159) | | |
| 12 | Reproductive (0.351) Cardiovascular (0.158) Nervous (0.105) | Cancer (0.509) Inflammation (0.175) Fetal (0.158) | pSPORT 1 |
| 13 | Reproductive (0.385) MusculoSkeletal (0.231) Nervous | Cancer (0.538) Inflammation (0.154) Other (0.154) | pSPORT 1 |
| | (0.154) | | |
| 14 | Endocrine (0.182) Gastrointestinal (0.182) MusculoSkeletal | Cancer (0.455) Fetal (0.273) Inflammation (0.273) | pINCY |
| | (0.182) | | |
| 15 | Reproductive (0.251) Nervous (0.186) Cardiovascular (0.150) | Cancer (0.545) Inflammation (0.174) Fetal (0.138) | pINCY |
| 16 | Hematopoietic/Immune (0.462) Reproductive (0.154) | Inflammation (0.462) Cancer (0.385) | pINCY |
| | Nervous (0.154) | | |

invention contemplates each and every possible variation of polynucleotide sequence that could be made by selecting combinations based on possible codon choices. These combinations are made in accordance with the standard triplet genetic code as applied to the polynucleotide sequence of naturally occurring REC, and all such variations are to be considered as being specifically disclosed.

Although nucleotide sequences which encode REC and its variants are preferably capable of hybridizing to the nucleotide sequence of the naturally occurring REC under appropriately selected conditions of stringency, it may be advantageous to produce nucleotide sequences encoding REC or its derivatives possessing a substantially different codon usage by inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding REC and its derivatives without altering the encoded amino acid sequences include the production of RNA transcripts having more desirable properties, such as a greater half-life, than transcripts produced from the naturally occurring sequence.

The invention also encompasses production of DNA sequences which encode REC and REC derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding REC or any fragment thereof.

Also encompassed by the invention are polynucleotide sequences that are capable of hybridizing to the claimed polynucleotide sequences, and, in particular, to those shown in SEQ ID NOs:17-32, and fragments thereof under various conditions of stringency. “Stringent conditions” refer to conditions which permit hybridization between polynucleotides. Stringent conditions can be defined by salt concentration, temperature, and other chemicals and conditions well known in the art. In particular, stringency can be increased by reducing the concentration of salt, or raising the hybridization temperature.

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Stringent temperature conditions will ordinarily include temperatures of at least about 30° C., more preferably of at least about 37° C., and most preferably of at least about 42° C. Varying additional parameters, such as hybridization time, the concentration of detergent (sodium dodecyl sulfate, SDS) or solvent (formamide), and the inclusion or exclusion of carrier DNA, are well known to those skilled in the art. Various levels of stringency are accomplished by combining these various conditions as needed. In a preferred embodiment, Southern hybridization will occur at 30° C. in 750 mM NaCl, 75 mM trisodium citrate, and 1% SDS. In a more preferred embodiment, Southern hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, Southern hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The stringency of washing steps which follow hybridization can also vary as defined by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York N.Y., and Sambrook, J. et al. (1989) Molecular Cloning, A Laboratory Manual, Cold Spring Harbor Press, Plainview N.Y.).

Methods for DNA sequencing are well known and generally available in the art and may be used to practice any of the embodiments of the invention. The methods may employ such enzymes as the Klenow fragment of DNA polymerase I, SEQUENASE® (Amersham Pharmacia Biotech, Piscataway N.J.), Taq polymerase (Perkin Elmer), thermostable T7 polymerase (Amersham Pharmacia Biotech), or combinations of polymerases and proofreading exonucleases such as those found in the ELONGASE Amplification System (Life Technologies, Gaithersburg Md.). Preferably, sequence preparation is automated with machines such as the the ABI Catalyst 800 (Perkin Elmer) or a Hamilton Micro Lab 2200 (Hamilton, Reno Nev.) in combination with thermal cyclers (for PCR). Sequencing is then carried out using either DNA sequencers (Perkin Elmer) or capillary electrophoresis (Molecular Dynamics).

The nucleotide and/or amino acid sequences of the Sequence Listing can be used to query sequences in the GenBank primate (pri), rodent (rod), and mammalian (mam), vertebrate (vrtp), and eukaryote (eukp) databases, SwissProt, BLOCKS (Bairoch, A. et al. (1997) Nucleic Acids Res. 25:217-221), PFAM, and other databases which contain previously identified and annotated motifs and sequences. Methods such as those which deal with primary sequence patterns and secondary structure gap penalties (Smith, T. et al. (1992) Protein Engineering 5:35-51) and programs and algorithms such as BLAST (Basic Local Alignment Search Tool; Altschul, S. F. (1993) J. Mol. Evol 36:290-300; and Altschul et al. (1990) J. Mol. Biol. 215:403-410), BLOCKS (Henikoff S. and Henikoff G. J. (1991) Nucleic Acids Research 19:6565-6572), Hidden Markov Models (HMM; Eddy, S. R. (1996; Cur. Opin. Str. Biol. 6:361-365) and Sonnhammer, E. L. L. et al. (1997; Proteins 28:405-420)), etc. can be used to manipulate and analyze nucleotide and amino acid sequences. These databases, programs, algorithms and other methods and tools are well known in the art and are described in Ausubel (supra, unit 7.7) and in Meyers, R. A. (1995; Molecular Biology and Biotechnology, Wiley VCH, Inc, New York N.Y., p 856-853).

The nucleic acid sequences encoding REC may be extended utilizing a partial nucleotide sequence and employing various PCR-based methods known in the art to detect upstream sequences, such as promoters and regulatory elements. (See, e.g., Dieffenbach, C. W. and G. S. Dveksler (1995; PCR Primer, a Laboratory Manual, Cold Spring Harbor Press, Plainview, N.Y., pp.1-5; Sarkar, G. (1993, PCR Methods Applic. 2:318-322); Triglia, T. et al. (1988; Nucleic Acids Res. 16:8186); Lagerstrom, M. et al. (1991; PCR Methods Applic. 1:111-119); and Parker, J. D. et al. (1991; Nucleic Acids Res. 19:3055-306). Additionally, one may use PCR, nested primers, and PROMOTERFINDER libraries to walk genomic DNA (Clontech, Palo Alto, Calif.). This procedure avoids the need to screen libraries and is useful in finding intron/exon junctions. For all PCR-based methods, primers may be designed using commercially available software, such as OLIGO 4.06 Primer Analysis software (National Biosciences Inc., Plymouth Minn.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Perkin Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode REC may be cloned in recombinant DNA molecules that direct expression of REC, or fragments or functional equivalents thereof, in appropriate host cells. Due to the inherent degeneracy of the genetic code, other DNA sequences which encode substantially the same or a functionally equivalent amino acid sequence may be produced and used to express REC.

The nucleotide sequences of the present invention can be engineered using methods generally known in the art in order to alter REC-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In another embodiment, sequences encoding REC may be synthesized, in whole or in part, using chemical methods well known in the art. (See, e.g., Caruthers, M. H. et al. (1980) Nucl. Acids Res. Symp. Ser. 215-223; Horn, T. et al. (1980) Nucl. Acids Res. Symp. Ser. 225-232; and Ausubel, supra) Alternatively, REC itself or a fragment thereof may be synthesized using chemical methods. For example, peptide synthesis can be performed using various solid-phase techniques (Roberge, J. Y. et al. (1995) Science 269:202-204). Automated synthesis may be achieved using the ABI 431A Peptide Synthesizer (Perkin Elmer). Additionally, the amino acid sequence of REC, or any part thereof, may be altered during direct synthesis and/or combined with sequences from other proteins, or any part thereof, to produce a variant polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Ausubel, supra) In order to express a biologically active REC, the nucleotide sequences encoding REC or, derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding REC. Such elements may vary in their strength and specificity. Specific initiation signals may also be used to achieve more efficient translation of sequences encoding REC. Such signals include the ATG initiation codon and adjacent sequences, e.g. the Kozak sequence. In cases where sequences encoding REC and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing sequences encoding REC and appropriate transcriptional and translational control elements. These methods include in vitro recombinant DNA techniques, synthetic techniques, and in vivo genetic recombination. (See, e.g., Sambrook (supra) and Ausubel, (supra) A variety of expression vector/host systems may be utilized to contain and express sequences encoding REC. These include, but are not limited to, microorganisms such as bacteria transformed with recombinant bacteriophage, plasmid, or cosmid DNA expression vectors; yeast transformed with yeast expression vectors; insect cell systems infected with viral expression vectors (baculovirus); plant cell systems transformed with viral expression vectors, cauliflower mosaic virus (CaMV) or tobacco mosaic virus (TMV), or with bacterial expression vectors (Ti or pBR322 plasmids); or animal cell systems. The invention is not limited by the host cell employed.

In bacterial systems, a number of cloning and expression vectors may be selected depending upon the use intended for polynucleotide sequences encoding REC. For example, routine cloning, subcloning, and propagation of polynucleotide sequences encoding REC can be achieved using a multifunctional E. coli vector such as BLUESCRIPT® (Stratagene, La Jolla Calif.) or pSPORT1 plasmid (Life Technologies). Ligation of sequences encoding REC into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of REC are needed, e.g. for the production of antibodies, vectors which direct high level expression of REC may be used. For example, vectors containing the strong, inducible T5 or T7 bacteriophage promoter may be used.

Yeast expression systems may be used for production of REC. A number of vectors containing constitutive or inducible promoters, such as alpha factor, alcohol oxidase, and PGH, may be used in the yeast Saccharomyces cerevisiae or Pichia pastoris. In addition, such vectors direct either the secretion or intracellular retention of expressed proteins and enable integration of foreign sequences into the host genome for stable propagation (Ausubel, supra; Scorer, C. A. et al. (1994) Bio/Technology 12:181-184).

Plant systems may also be used for expression of REC. Transcription of sequences encoding REC may be driven by viral promoters such as the ³⁵S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. (See McGraw Hill Yearbook of Science and Technology (1992) McGraw Hill, New York N.Y.; pp. 191-196.) In mammalian cells, a number of viral-based expression systems may be utilized. In cases where an adenovirus is used as an expression vector, sequences encoding REC may be ligated into an adenovirus transcription/translation complex consisting of the late promoter and tripartite leader sequence. Insertion in a non-essential E1 or E3 region of the viral genome may be used to obtain infective virus which expresses REC in host cells (Logan, J. and T. Shenk (1984) Proc. Natl. Acad. Sci. 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes.

For long term production of recombinant proteins in mammalian systems, stable expression of REC in cell lines is preferred. For example, sequences encoding REC can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ or apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als or pat confer resistance to chlorsulfuron and phosphinotricin acetyltransferase, respectively. Additional selectable genes have been described, e.g., trpB and hisD, which alter cellular requirements for metabolites (Hartman, S. C. and R. C. Mulligan (1988) Proc. Natl. Acad. Sci. 85:8047-8051). Visible markers, e.g., anthocyanins, green fluorescent proteins (GFP; Clontech, Palo Alto, Calif.), B glucuronidase and its substrate B-D-glucuronide, or luciferase and its substrate luciferin may be used. These markers can be used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, the presence and expression of the gene may need to be confirmed. For example, if the sequence encoding REC is inserted within a marker gene sequence, transformed cells containing sequences encoding REC can be identified by the absence of marker gene function. Alternatively, a marker gene can be placed in tandem with a sequence encoding REC under the control of a single promoter. Expression of the marker gene in response to induction or selection usually indicates expression of the tandem gene as well.

In general, host cells that contain the nucleic acid sequence encoding REC and that express REC may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of REC using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on REC is preferred, but a competitive binding assay may be employed. These and other assays are well known in the art. (See, e.g., Hampton, R. et al. (1990) Serological Methods, a Laboratory Manual, APS Press, St Paul, Minn., Section IV; Coligan, J. E. et al. (1997 and periodic supplements) Current Protocols in Immunology, Greene Pub. Associates and Wiley-Interscience, New York, N.Y.; and Maddox, D. E. et al. (1983) J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding REC include oligolabeling, nick translation, end-labeling, or PCR amplification using a labeled nucleotide. Alternatively, the sequences encoding REC, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding REC may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode REC may be designed to contain signal sequences which direct secretion of REC through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and W138), are available from the American Type Culture Collection (ATCC, Bethesda, Md.) and may be chosen to ensure the correct modification and processing of the foreign protein.

In another embodiment of the invention, natural, modified, or recombinant nucleic acid sequences encoding REC may be ligated to a heterologous sequence resulting in translation of a fusion protein in any of the aforementioned host systems. For example, a chimeric REC protein containing a heterologous moiety that can be recognized by a commercially available antibody may facilitate the screening of peptide libraries for inhibitors of REC activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the REC encoding sequence and the heterologous protein sequence, so that REC may be cleaved away from the heterologous moiety following purification. Methods for fusion protein expression and purification are discussed in Ausubel (supra). A variety of commercially available kits may also be used to facilitate expression and purification of fusion proteins.

In a further embodiment of the invention, synthesis of radiolabeled REC may be achieved in vitro using the TNT rabbit reticulocyte lysate or wheat germ extract systems (Promega). These systems couple transcription and translation of protein-coding sequences operably associated with the T7, T3, or SP6 promoters. Translation takes place in the presence of a radiolabeled amino acid precursor, preferably ³⁵S-methionine.

Fragments of REC may be produced not only by recombinant production, but also by direct peptide synthesis using solid-phase techniques (Creighton, supra pp. 55-60). Protein synthesis may be performed by manual techniques or by automation. Automated synthesis may be achieved, for example, using the ABI 431A Peptide Synthesizer (Perkin Elmer). Various fragments of REC may be synthesized separately and then combined to produce the full length molecule.

Therapeutics

Chemical and structural similarity exists among REC and receptor molecules. In addition, REC is expressed in cancer, immunological, fetal, reproductive, gastrointestinal, nervous, smooth muscle, endocrine, and musculoskeletal tissues. Therefore, REC appears to play a role in neoplastic, immunological, reproductive, gastrointestinal, nervous, smooth muscle, and musculoskeletal disorders.

Therefore, in one embodiment, an antagonist of REC may be administered to a subject to treat or prevent a neoplastic disorder. Such a neoplastic disorder may include, but is not limited to, adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a neoplastic disorder including, but not limited to, those described above.

In a further embodiment, an antagonist of REC may be administered to a subject to treat or prevent an immunological disorder. Such an immunological disorder may include, but is not limited to, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Wemer syndrome, complications of cancer, hemodialysis, and extracorporeal circulation, viral, bacterial, fungal, parasitic, protozoal, and helminthic infections, and trauma. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent an immunological disorder including, but not limited to, those described above.

In a further embodiment, an antagonist of REC may be administered to a subject to treat or prevent a reproductive disorder. Such a reproductive disorder may include, but is not limited to, disorders of prolactin production; infertility, including tubal disease, ovulatory defects, and endometriosis; disruptions of the estrous cycle, disruptions of the menstrual cycle, polycystic ovary syndrome, ovarian hyperstimulation syndrome, endometrial and ovarian tumors, uterine fibroids, autoimmune disorders, ectopic pregnancies, and teratogenesis; cancer of the breast, fibrocystic breast disease, and galactorrhea; disruptions of spermatogenesis, abnormal sperm physiology, cancer of the testis, cancer of the prostate, benign prostatic hyperplasia, prostatitis, Peyronie's disease, carcinoma of the male breast, and gynecomastia. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a reproductive disorder including, but not limited to, those described above.

In a further embodiment, an antagonist of REC may be administered to a subject to treat or prevent a gastrointestinal disorder. Such a gastrointestinal disorder may include, but is not limited to, dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, and acquired immunodeficiency syndrome (AIDS) enteropathy, cirrhosis, jaundice, cholestasis, hereditary hyperbilirubinemia, hepatic encephalopathy, hepatorenal syndrome, hepatitis, hepatic steatosis, hemochromatosis, Wilson's disease, α₁-antitrypsin deficiency, Reye's syndrome, primary sclerosing cholangitis, liver infarction, portal vein obstruction and thrombosis, passive congestion, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a gastrointestinal disorder including, but not limited to, those described above.

In a further embodiment, an antagonist of REC may be administered to a subject to treat or prevent a nervous disorder. Such a nervous disorder may include, but is not limited to, akathesia, Alzheimer's disease, amnesia, amyotrophic lateral sclerosis, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, Parkinson's disease, paranoid psychoses, postherpetic neuralgia, schizophrenia, and Tourette's disorder. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a nervous disorder including, but not limited to, those described above.

In a further embodiment, an antagonist of REC may be administered to a subject to treat or prevent a smooth muscle disorder. A smooth muscle disorder is defined as any impairment or alteration in the normal action of smooth muscle and may include, but is not limited to, angina, anaphylactic shock, arrhythmias, asthma, cardiovascular shock, Cushing's syndrome, hypertension, hypoglycemia, myocardial infarction, migraine, and pheochromocytoma, and myopathies including cardiomyopathy, encephalopathy, epilepsy, Kearns-Sayre syndrome, lactic acidosis, myoclonic disorder, and ophthalmoplegia. Smooth muscle includes, but is not limited to, that of the blood vessels, gastrointestinal tract, heart, and uterus. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a smooth muscle disorder including, but not limited to, those described above.

In a further embodiment, an antagonist of REC may be administered to a subject to treat or prevent a musculoskeletal disorder. Such a musculoskaletal disorder may include, but is not limited to, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, and ethanol myopathy. In one aspect, an antibody which specifically binds REC may be used directly as an antagonist or indirectly as a targeting or delivery mechanism for bringing a pharmaceutical agent to cells or tissue which express REC.

In an additional embodiment, a vector expressing the complement of the polynucleotide encoding REC may be administered to a subject to treat or prevent a musculoskeletal disorder including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of REC may be produced using methods which are generally known in the art. In particular, purified REC may be used to produce antibodies or to screen libraries of pharmaceutical agents to identify those which specifically bind REC. Antibodies to REC may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others may be immunized by injection with REC or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to REC have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of REC amino acids may be fused with those of another protein, such as KLH, and antibodies to the chimeric molecule may be produced.

Monoclonal antibodies to REC may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495497; Kozbor, D. et al. (1985) J. Immunol. Methods 81:3142; Cote, R. J. et al. (1983) Proc. Natl. Acad. Sci. 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce REC-specific single chain antibodies. Antibodies with related specificity, but of distinct idiotypic composition, may be generated by chain shuffling from random combinatorial immunoglobulin libraries (Burton D. R. (1991) Proc. Natl. Acad. Sci. 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites for REC may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between REC and its specific antibody. A two-site, monoclonal-based immunoassay utilizing monoclonal antibodies reactive to two non-interfering REC epitopes is preferred, but a competitive binding assay may also be employed (Maddox, supra).

In another embodiment of the invention, the polynucleotides encoding REC, or any fragment or complement thereof, may be used for therapeutic purposes. In one aspect, the complement of the polynucleotide encoding REC may be used in situations in which it would be desirable to block the transcription of the mRNA. In particular, cells may be transformed with sequences complementary to polynucleotides encoding REC. Thus, complementary molecules or fragments may be used to modulate REC activity, or to achieve regulation of gene function. Such technology is now well known in the art, and sense or antisense oligonucleotides or larger fragments can be designed from various locations along the coding or control regions of sequences encoding REC.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding REC (Sambrook, supra; Ausubel, supra).

Genes encoding REC can be turned off by transforming a cell or tissue with expression vectors which express high levels of a polynucleotide, or fragment thereof, encoding REC. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding REC. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing Co., Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding REC.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding REC. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as

acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nature Biotechnology 15:462-466.)

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical or sterile composition, in conjunction with a pharmaceutically acceptable carrier, for any of the therapeutic effects discussed above. Such pharmaceutical compositions may consist of REC, antibodies to REC, and mimetics, agonists, antagonists, or inhibitors of REC. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of REC, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example REC or fragments thereof, antibodies of REC, and agonists, antagonists or inhibitors of REC, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of therapeutic to toxic effects is the therapeutic index, and it can be expressed as the ED₅₀/LD₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind REC may be used for the diagnosis of disorders characterized by expression of REC, or in assays to monitor patients being treated with REC or agonists, antagonists, or inhibitors of REC. Antibodies useful for diagnostic purposes may be prepared in the same manner as described above for therapeutics. Diagnostic assays for REC include methods which utilize the antibody and a label to detect REC in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring REC, including ELISAs, RIAs, and FACS, are known in the art and provide a basis for diagnosing altered or abnormal levels of REC expression. Normal or standard values for REC expression are established by combining body fluids or cell extracts taken from normal mammalian subjects, preferably human, with antibody to REC under conditions suitable for complex formation The amount of standard complex formation may be quantitated by various methods, preferably by photometric means. Quantities of REC expressed in subject, control, and disease samples from biopsied tissues are compared with the standard values. Deviation between standard and subject values establishes the parameters for diagnosing disease.

In another embodiment of the invention, the polynucleotides encoding REC may be used for diagnostic purposes. The polynucleotides which may be used include oligonucleotide sequences, complementary RNA and DNA molecules, and PNAs (Nielsen, P. E. et al. (1993) Anticancer Drug Des. 8:53-63). The polynucleotides may be used to detect and quantitate gene expression in biopsied tissues in which expression of REC may be correlated with disease. The diagnostic assay may be used to determine absence, presence, and excess expression of REC, and to monitor regulation of REC levels during therapeutic intervention.

In one aspect, hybridization with PCR probes which are capable of detecting polynucleotide sequences, including genomic sequences, encoding REC or closely related molecules may be used to identify nucleic acid sequences which encode REC. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding REC, allelic variants, or related sequences.

Probes may also be used for the detection of related sequences, and should preferably have at least 50% sequence identity to any of the REC encoding sequences. The hybridization probes of the subject invention may be DNA or RNA and may be derived from the sequence of SEQ ID NOs:17-32, or from genomic sequences including promoters, enhancers, and introns of the REC gene.

Means for producing specific hybridization probes for DNAs encoding REC include the cloning of polynucleotide sequences encoding REC or REC derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding REC may be used for the diagnosis of a disorder associated with expression of REC. Examples of such a disorder include, but are not limited to, a neoplastic disorder, such as, adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an immunological disorder, such as, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Wemer syndrome, complications of cancer, hemodialysis, and extracorporeal circulation, viral, bacterial, fungal, parasitic, protozoal, and helminthic infections, and trauma; a reproductive disorder, such as, disorders of prolactin production; infertility, including tubal disease, ovulatory defects, and endometriosis; disruptions of the estrous cycle, disruptions of the menstrual cycle, polycystic ovary syndrome, ovarian hyperstimulation syndrome, endometrial and ovarian tumors, uterine fibroids, autoimmune disorders, ectopic pregnancies, and teratogenesis; cancer of the breast, fibrocystic breast disease, and galactorrhea; disruptions of spermatogenesis, abnormal sperm physiology, cancer of the testis, cancer of the prostate, benign prostatic hyperplasia, prostatitis, Peyronie's disease, carcinoma of the male breast, and gynecomastia; a gastrointestinal disorder, such as, dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, and acquired immunodeficiency syndrome (AIDS) enteropathy, cirrhosis, jaundice, cholestasis, hereditary hyperbilirubinemia, hepatic encephalopathy, hepatorenal syndrome, hepatitis, hepatic steatosis, hemochromatosis, Wilson's disease, alpha₁-antitrypsin deficiency, Reye's syndrome, primary sclerosing cholangitis, liver infarction, portal vein obstruction and thrombosis, passive congestion, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a nervous disorder, such as, akathesia, Alzheimer's disease, amnesia, amyotrophic lateral sclerosis, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, Parkinson's disease, paranoid psychoses, postherpetic neuralgia, schizophrenia, and Tourette's disorder; a smooth muscle disorder, such as, angina, anaphylactic shock, arrhythmias, asthma, cardiovascular shock, Cushing's syndrome, hypertension, hypoglycemia, myocardial infarction, migraine, and pheochromocytoma, and myopathies including cardiomyopathy, encephalopathy, epilepsy, Kearns-Sayre syndrome, lactic acidosis, myoclonic disorder, and ophthalmoplegia. Smooth muscle includes, but is not limited to, that of the blood vessels, gastrointestinal tract, heart, and uterus; and a musculoskaletal disorder, such as, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, and ethanol myopathy. The polynucleotide sequences encoding REC may be used in Southern or Northern analysis, dot blot, or other membrane-based technologies; in PCR technologies; in dipstick, pin, and ELISA assays; and in microarrays utilizing fluids or tissues from patients to detect altered REC expression. Such qualitative or quantitative methods are well known in the art.

In a particular aspect, the nucleotide sequences encoding REC may be useful in assays that detect the presence of associated disorders, particularly those mentioned above. The nucleotide sequences encoding REC may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding REC in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of REC, a normal or standard profile for expression is established. This may be accomplished by combining body fluids or cell extracts taken from normal subjects, either animal or human, with a sequence, or a fragment thereof, encoding REC, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding REC may involve the use of PCR. These oligomers may be chemically synthesized, generated enzymatically, or produced in vitro. Oligomers will preferably contain a fragment of a polynucleotide encoding REC, or a fragment of a polynucleotide complementary to the polynucleotide encoding REC, and will be employed under optimized conditions for identification of a specific gene or condition. Oligomers may also be employed under less stringent conditions for detection or quantitation of closely related DNA or RNA sequences.

Methods which may also be used to quantitate the expression of REC include radiolabeling or biotinylating nucleotides, coamplification of a control nucleic acid, and interpolating results from standard curves. (See, e.g., Melby, P. C. et al. (1993) J. Immunol. Methods 159:235-244; and Duplaa, C. et al. (1993) Anal. Biochem. 229-236.) The speed of quantitation of multiple samples may be accelerated by running the assay in an ELISA format where the oligomer of interest is presented in various dilutions and a spectrophotometric or calorimetric response gives rapid quantitation.

In further embodiments, oligonucleotides or longer fragments derived from any of the polynucleotide sequences of the Sequence Listing may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.)

In another embodiment of the invention, nucleic acid sequences encoding REC may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Price, C. M. (1993) Blood Rev. 7:127-134; Trask, B. J. (1991) Trends Genet. 7:149-154).

Fluorescent in situ hybridization (FISH) may be correlated with other physical chromosome mapping techniques and genetic map data (Heinz-Ulrich, et al. (1995) in Meyers, R. A. (ed.) Molecular Biology and Biotechnology, VCH Publishers New York N.Y., pp. 965-968). Examples of genetic map data can be found in various scientific journals or at the Online Mendelian Inheritance in Man (OMIM) site. Correlation between the location of the gene encoding REC on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, REC, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between REC and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate, such as plastic pins or some other surface. The test compounds are reacted with REC, or fragments thereof, and washed. Bound REC is then detected by methods well known in the art. Purified REC can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding REC specifically compete with a test compound for binding REC. In this manner, antibodies can be used to detect the presence of any peptide which shares one or more antigenic determinants with REC.

In additional embodiments, the nucleotide sequences which encode REC may be used in any molecular biology techniques that have yet to be developed, provided the new techniques rely on properties of nucleotide sequences that are currently known, including, but not limited to, such properties as the triplet genetic code and specific base pair interactions.

The examples below are provided to illustrate the subject invention and are not included for the purpose of limiting the invention.

### EXAMPLES

I. cDNA Library Construction

Tissue Description

The TBLYNOT01 library was constructed at Stratagene (STR937214) using RNA isolated from a hybrid of T-B lymphoblasts from a leukemic cell line.

The HNT2NOT01 library was constructed at Stratagene (STR937230) using RNA isolated from the hNT2 cell line derived from a human teratocarcinoma that exhibited properties characteristic of a committed neuronal precursor at an early stage of development.

The PROSTUT05 library was constructed using polyA RNA isolated from prostate tumor tissue removed from a 69-year-old Caucasian male. Pathology indicated adenofibromatous hyperplasia and adenocarcinoma (Gleason grade 3 and 4). The tumor invaded the capsule but did not extend beyond it; perineural invasion was present. The patient presented with elevated prostate specific antigen. Patient history included occlusion of a leg vein, diverticuli of the colon, and a partial colectomy. Family history included cardiovascular disease, multiple myeloma, hyperlipidemia, and rheumatoid arthritis.

The BRSTNOT05 library was constructed using polyA RNA isolated from nontumorous breast tissue removed from a 58-year-old Caucasian female. Pathology for the associated tumor tissue indicated multicentric invasive grade 4 lobular carcinoma. Patient history included skin cancer, rheumatic heart disease, osteoarthritis, and tuberculosis. Family history included cerebrovascular and cardiovascular disease, breast and prostate cancer, and Type I diabetes.

The THYRNOT03 library was constructed using polyA RNA isolated from thyroid tissue removed from a 28-year-old Caucasian female. Pathology indicated adenomatous hyperplasia associated with follicular adenoma. Patient history included nonobstetrical galactorrhea, anemia, and pure hypercholesterolemia. Family history included hyperlipidemia skin cancer, and neurotic depression.

The LUNGNOT14 library was constructed using polyA RNA isolated from nontumorous lung tissue removed from a 47-year-old Caucasian male during a segmental lung resection. Pathology of the associated tumor indicated a grade 4 adenocarcinoma and calcified granuloma. Patient history included benign hypertension and chronic obstructive pulmonary disease. Family history included cardiovascular disease, and Type II diabetes.

The CONNNOT01 library was constructed using polyA RNA isolated from mesentery fat tissue removed from a 71-year-old Caucasian male during a partial colectomy. Patient history included a diverticulosis and diverticulitis, cholecystectomy, viral hepatitis, and a hemagioma. The patient was taking Tegretol (carbamazepine). Family history included cardiovascular disease and extrinsic asthma.

The KERANOT02 library was constructed using polyA RNA isolated from human breast keratinocyte cell line (NHEK, Clontech).

The BEPINOT01 library was constructed using polyA RNA isolated from a bronchial epithelium primary cell line derived from a 54-year-old Caucasian male (NHBE, Clontech).

The BRSTNOT07 library was constructed using polyA RNA isolated from nontumorous breast tissue removed from a 43-year-old Caucasian female. Pathology indicated mildly proliferative fibrocystic changes with epithelial hyperplasia, papillomatosis, and duct ectasia. The associated tumor tissue indicated invasive, grade 4 mammary adenocarcinoma with extensive comedo necrosis. Family history included cardiovascular disease; epilepsy, and Type II diabetes.

The OVARNOT03 library was constructed using polyA RNA isolated from nontumorous ovarian tissue removed from a 43-year-old Caucasian female. Pathology for the associated tumor tissue indicated grade 2 mucinous cystadenocarcinoma. Patient history included mitral valve disorder, pneumonia, and viral hepatitis. Family history included cardiovascular and cerebrovascular disease and pancreatic, breast, and uterine cancer.

The OVARNOT02 library was constructed using polyA RNA isolated from ovarian tissue removed from a 59-year-old Caucasian female who died of a myocardial infarction. Patient history included cardiovascular disease, hypercholesterolemia, hypotension, and arthritis.

The ADRETUT06 library was constructed using polyA RNA isolated from adrenal tumor tissue removed from a 57-year-old Caucasian female. Pathology indicated pheochromocytoma. Patient history included cardiovascular and cerebrovascular disease, type I diabetes, reflux esophagitis, and joint pain. Family history included cardiovascular disease, type I diabetes, renal failure, and skin cancer.

The THYMFET02 library was constructed using polyA RNA isolated from thymus tissue removed from a Caucasian female fetus who died at 17 weeks' gestation from anencephaly.

The SKINNOT04 library was constructed using polyA RNA isolated from breast skin tissue removed from a 70-year-old Caucasian female during a biopsy and resection. Pathology for the associated tumor tissue indicated invasive grade 3 adenocarcinoma.

mRNA Isolation and Library Construction

RNA was purchased from Clontech (Palo Alto, Calif.) or isolated at Incyte from the tissues described above. The tissue was homogenized, lysed, and extracted in phenol, guanidinium isothiocyanate, or a suitable mixture of denaturants such as TRIZOL reagent (Life Technologies), a monophasic solution of phenol and guanidine isothiocyanate. To isolate RNA, lysate was centrifuged over a Csc cushion, mixed with chloroform (1:5 v/v), recovered in the aqueous phase and precipitated with isopropanol. Alternatively, lysate was electrophoresed through an agarose gel, and RNA was collected using Whitman P81 paper (Whitman, Lexington Mass.) and eluted. The eluted RNA was precipitated with sodium acetate and ethanol. The precipitant was resuspended in RNase-free water. For some libraries, RNA was treated with DNase; and for others, phenol extraction and precipitation were repeated. For most libraries, poly(A+) RNA was isolated using oligo d(T)-coupled paramagnetic particles (Promega), Oligotex resin or the Oligotex kit (QIAGEN Inc, Chatsworth, Calif.), or the Stratagene RNA Isolation kit. Alternatively, RNA was isolated directly from tissue lysates using the Ambion PolyA Quick kit (Ambion, Austin, Tex.).

The cDNA libraries were synthesized and constructed at Stratagene or at Incyte according to procedures recommended in the UNIZAP vector (Stratagene) or SUPERSCRIPT plasmid system (Life Technologies), both of which are based on methods well known in the art (Ausubel, supra, units 5.1-6.6). Alternatively, cDNA libraries were constructed by Stratagene using RNA provided by Incyte. Reverse transcription was initiated using oligo d(T) or random primers. Synthetic oligonucleotide adapters were ligated to double stranded cDNA, and cDNA was digested with an appropriate restriction enzyme(s). For most libraries, cDNA was size-selected (300-1000 bp) using Sephacryl S1000 or Sepharose CL2B or CL4B column chromatography (Amersham Pharmacia Biotech) or preparative agarose gel electrophoresis. cDNAs were ligated into compatible restriction enzyme site of the polylinker of a suitable plasmid, e.g., pBLUESCRIPT (Stratagene), pSPORT 1 (Life Technologies), pINCY1 (Incyte Pharmaceuticals Inc, Palo Alto, Calif.). pINCY1 was amplified in JM109 cells and purified using the QiaQuick column (QIAGEN Inc). Recombinant plasmids were transformed into competent E. coli cells, e.g., XL1-Blue, XL1-BlueMRF, or SOLR (Stratagene) or DH5α, DH10B, or ElectroMAX DH10B (Life Technologies).

III. Sequencing and Analysis

The cDNAs were prepared for sequencing using either an ABI Catalyst 800 (Perkin Elmer) or a Hamilton Micro Lab 2200 (Hamilton, Reno, Nev.) in combination with Peltier Thermal Cyclers (PTC200; MJ Research, Watertown Mass.). The cDNAs were sequenced on the ABI 373 or 377 DNA Sequencing systems (Perkin Elmer) by the method of Sanger et al. (1975; J. Mol. Biol. 94:441f) using stardard ABI protocols and kits. In the alternative, cDNAs may have been sequenced using solutions and dyes from Amersham Pharmacia Biotech. Reading frame was determined using standard methods (Ausubel, supra).

The cDNA sequences presented by Incyte Clone number in the last column of Table 1 and the full length nucleotide and amino acid sequences disclosed in the Sequence Listing were analyzed and characterized using several of the following programs (or algorithms) and databases. For PFAM, scores >11 report a significant degree of correlation; and the higher the value, the more homologous the query sequence is to members of the protein family. HMM models which were used to identify and confirm signal sequences (SIGPEPT), transmembrane domains (TM) and the receptors disclosed in the Sequence Listing were developed with annotated sequences from LIFESEQ® database (Incyte Pharmaceuticals, Palo Alto Calif.) and SwissProt database. BLAST and the derivation of product score are described in example IV below.

| Program/algorithm | Databases | Description | Useful Parameters |
|---|---|---|---|
| cDNAs | | | |
| Smith Waterman | GenBank | Local alignment algorithm | min length = 49 nt |
| | | for homology searching | <12% uncalled bases |
| FASTA | GenBank | Fast nucleotide sequence | |
| | | database searching program | |
| | | for UNIX, VMS | |
| BLAST | GenBank | Ultra-fast database | Log likelihood for exact |
| | | searching program for | matches is 10⁻²⁵ and for |
| | | UNIX, VMS C source | homologs > 10⁻⁸ |
| Full Length | | | |
| Phred | | Reads trace data from | |
| | | sequencing runs, makes base | |
| | | calls, produces quality scores | |
| | | and DNA sequence | |
| Phlame | | Reads trace data from | |
| | | sequencing runs, makes base | |
| | | calls, produces quality scores | |
| | | and DNA sequence | |
| Phrap | | Quality-score based | match > 56 |
| | | assembly program for shotgun | score > 120 |
| | | sequences | |
| CONSED | | Graphical tool for editing | |
| | | PHRAP contigs | |
| BLAST | GenBank | Ultra-fast database searching | score >100 |
| | SwissProt | program for UNIX, VMS | P < 1e−5 |
| | | C source | |
| FASTX | GenBank | Fast amino acid sequence | log likelihood > 17 |
| | SwissProt | database searching program | |
| | | for UNIX, VMS | |
| BLIMPS | BLOCKS | Weighted-matrix analyses used | >1300 strong |
| | PRINTS | to predict protein classification | 1000-1300 suggestive |
| | | | P < 1e−3 |
| PFAM | PROSITE | Analyses 3-60 amino acid | >11 strong |
| | | long sequences which | 8-10 suggestive |
| | | correspond to highly | |
| | | conserved regions of a | |
| | | protein family | |
| HMM | SwissProt | Hidden Markov Models analyze | Score > 11 |
| (SIGPEPT, | | primary structures of gene families | |
| TM, and | | using probabilistic approaches | |
| Receptor) | | and trained models | |

IV. Northern Analysis

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7).

Analogous computer techniques applying BLAST are used to search for identical or related molecules in nucleotide databases such as GenBank or LIFESEQ® database (Incyte Pharmaceuticals). This analysis is much faster than multiple membrane-based hybridizations. In addition, the sensitivity of the computer search can be modified to determine whether any particular match is categorized as exact or similar.

The basis of the search is the product score, which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1% to 2% error, and, with a product score of 70, the match will be exact. Similar molecules are usually identified by selecting those which show product scores between 15 and 40, although lower scores may identify related molecules.

The results of Northern analysis are reported as a list of libraries in which the transcript encoding REC occurs. Abundance and percent abundance are also reported. Abundance directly reflects the number of times a particular transcript is represented in a cDNA library, and percent abundance is abundance divided by the total number of sequences examined in the cDNA library.

V. Extension of REC Encoding Polynucleotides

The nucleic acid sequences of Incyte Clones 044150, 266775, 843183, 965938, 1441620, 1510911, 2022379, 2024312, 2057886, 2121924, 2122815, 2132179, 2326441, 2825826, 2936050, and 3428945 were used to design oligonucleotide primers for extending partial nucleotide sequences to full length. For each nucleic acid sequence, one primer was synthesized to initiate extension of an antisense polynucleotide, and the other was synthesized to initiate extension of a sense polynucleotide. Primers were used to facilitate the extension of the known sequence “outward” generating amplicons containing new unknown nucleotide sequence for the region of interest. The initial primers were designed from the cDNA using OLIGO 4.06 (National Biosciences, Plymouth, Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries (Life Technologies) were used to extend the sequence. If more than one extension is necessary or desired, additional sets of primers are designed to further extend the known region.

High fidelity amplification was obtained by following the instructions for the XL-PCR kit (Perkin Elmer) and thoroughly mixing the enzyme and reaction mix. PCR was performed using the Peltier Thermal Cycler (MJ Research), beginning with 40 pmol of each primer and the recommended concentrations of all other components of the kit, with the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat steps 4 through 6 for an additional 15 cycles |
| Step 8 | 94° C. for 15 sec |
| Step 9 | 65° C. for 1 min |
| Step 10 | 68° C. for 7:15 min |
| Step 11 | Repeat steps 8 through 10 for an additional 12 cycles |
| Step 12 | 72° C. for 8 min |
| Step 13 | 4° C. (and holding) |

A 5 μl to 10 μl aliquot of the reaction mixture was analyzed by electrophoresis on a low concentration (about 0.6% to 0.8%) agarose mini-gel to determine which reactions were successful in extending the sequence. Bands thought to contain the largest products were excised from the gel, purified using QIAQUICK (QIAGEN Inc.), and trimmed of overhangs using Klenow enzyme to facilitate religation and cloning.

After ethanol precipitation, the products were redissolved in 13 μl of ligation buffer, 1 μl T4-DNA ligase (15 units) and 1 μl T4 polynucleotide kinase were added, and the mixture was incubated at room temperature for 2 to 3 hours, or overnight at 16° C. Competent E. coli cells (in 40 μl of appropriate media) were transformed with 3 μl of ligation mixture and cultured in 80 μl of SOC medium. (See, e.g., Sambrook, supra, Appendix A, p. 2.) After incubation for one hour at 37° C., the E. coli mixture was plated on Luria Bertani (LB) agar (See, e.g., Sambrook, supra, Appendix A, p. 1) containing carbenicillin (2×carb). The following day, several colonies were randomly picked from each plate and cultured in 150 μl of liquid LB/2×carb medium placed in an individual well of an appropriate commercially-available sterile 96-well microtiter plate. The following day, 5 μl of each overnight culture was transferred into a non-sterile 96-well plate and, after dilution 1:10 with water, 5 μl from each sample was transferred into a PCR array.

For PCR amplification, 18 μl of concentrated PCR reaction mix (3.3×) containing 4 units of rTth DNA polymerase, a vector primer, and one or both of the gene specific primers used for the extension reaction were added to each well. Amplification was performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2 through 4 for an additional 29 cycles |
| Step 6 | 72° C. for 180 sec |
| Step 7 | 4° C. (and holding) |

Aliquots of the PCR reactions were run on agarose gels together with molecular weight markers. The sizes of the PCR products were compared to the original partial cDNAs, and appropriate clones were selected, ligated into plasmid, and sequenced.

In like manner, the nucleotide sequence of SEQ ID NOs: 17-32, are used to obtain 5′ regulatory sequences using the procedure above, oligonucleotides designed for 5′ extension, and an appropriate genomic library.

VI. Labeling and Use of Individual Hybridization Probes

Hybridization probes derived from SEQ ID NOs:17-32 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN®, Boston, Mass.). The labeled oligonucleotides are substantially purified using a SEPHADEX G-25 superfine size exclusion dextran bead column (Pharmacia & Upjohn, Kalamazoo, Mich.). An aliquot containing 10⁷ counts per minute of the labeled probe is used in a typical membrane-based hybridization analysis of human genomic DNA digested with one of the following endonucleases: Ase I, Bgl II, Eco RI, Pst I, XbaI, or Pvu II (DuPont NEN, Boston, Mass.).

The DNA from each digest is fractionated on a 0.7% agarose gel and transferred to nylon membranes (Nytran Plus, Schleicher & Schuell, Durham, N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMAT AR film (Kodak, Rochester, N.Y.) is exposed to the blots to film for several hours, hybridization patterns are compared visually.

VII. Microarrays

Membrane Preparation

A single 22×22 cm nylon membrane suitable for standard hybridization protocols is spotted with plant cDNA clones as follows. The clones are robotically picked and arrayed into 384-well culture dishes. The cultures are gridded, using a Q-Bot robot (Genetix Ltd), onto the nylon membrane at a density of 36,864 spots per membrane or 18,394 individual genes and 38 controls spotted in duplicate. These membranes are used in standard hybridization protocols described below.

Several membranes are placed on LB plates with carbenicillin in bioassay trays and grown for about 16 hours at 42° C. after which the membranes are placed (colony side up) for 4 minutes on top of Whatman filter paper (Whatman Inc, Lexington Mass.) previously saturated with prewarmed (95° C. to 100° C.) denaturing buffer (1.5M NaCl, 0.5M NaOH). Excess denaturing buffer is removed, and the membranes are saturated for 4 minutes with neutralizing buffer (1.5M NaCl, 1M Tris (Tris[hydroxymethyl]aminomethane) pH 8.0) by placing them (colony side up) on top of Whatman filter paper (Whatman, Inc) previously saturated with neutralizing buffer. The membranes are dried until no liquid is visible on their surfaces.

Next the membranes are submerged, colony side down, in 100 ml prewarmed (42° C.) proteinase K buffer which consists of 0.1 M NaCl, 50 mM EDTA pH 8.5, 50 mM Tris pH 8.0, Sarkosyl (1% N-lauroyl sarcosine), and 1 mg/ml proteinase K (Sigma). After one hour, the membranes are retrieved and placed on Whatman filter paper (Whatman, Inc) to dry overnight. Finally, the membranes are exposed to UV light (254 nm for 40 seconds) in a GS Gene Linker UV Chamber (Bio-Rad Laboratories, Hercules Calif.) which cross-links the DNA to the membranes.

Probe Preparation

Five μg mRNA and 2 μl random hexamer (0.5 mg/ml; Life Technologies) are combined in a 1.5 ml RNase-free microcentrifuge tube. The sample is incubated at 70° C. for 10 minutes, placed on ice for five minutes, lyophilized to dryness, and then dissolved in the following: 1.6 μl 5×first strand buffer, 0.8 μl 0.1 M DTT, 0.4 μl 10 mM dA/dG/dT mix, 4.0 μl [³²P] dCTP (3000 Ci/mmol, 10 uCi/μl) and 1.2 μl SuperScript II RT (200 U/μl; Life Technologies).

The sample is centrifuged and incubated at 42° C. for 1 to 2 hours and then diluted with 42 μl of sterile water. Unincorporated nucleotides are removed with a ProbeQuant G-50 Microcolumn (Amersham Pharmacia Biotech). The purified sample is boiled at 95° C. for 3 minutes and then put on ice. To degrade mRNA, 12.5 μl of 1N NaOH are added to the sample which then is incubated at 37° C. for 10 minutes. The sample is neutralized by addition of 12.5 μl 1M Tris pH 6.8 and 10 μl 1M HCl. Degraded RNA is removed with a ProbeQuant G-50 Microcolumn (supra).

Hybridization

The hybridization procedure described by Soares is followed (Soares et al. Proc. Natl. Acad. Sci. (1994) 91:9228-9232). Ten mls prewarmed (42° C.) hybridization buffer (0.75 M NaCl, 0.1 M NaPO₄, 0.1% (w/v) NaP₂O₇, 0.15 M Tris pH 7.5, 5× Denhardt solution (Ausubel, supra), 2% sodium dodecyl sulfate (SDS), sheared salmon testes DNA (100 μg/ml), 50% formamide) are added to the membranes in hybridization bags for greater than 2 hours to overnight for prehybridization. Radiolabelled probe (³²P) is added to a new 10 ml aliquot of the prewarmed hybridization buffer, and hybridization is allowed to proceed at 42° C. for 14 to 16 hours.

After hybridization, membranes are rinsed with 200 ml 2×SSC at room temperature for 5 minutes, washed once with prewarmed 2×SSC plus 1% SDS for 20 minutes at 68° C., and then washed two more times with prewarmed 0.6×SSC plus 1% SDS for 30 minutes at 68° C. Damp membranes are exposed to X-OMAT autoradiography film (Kodak) for two nights in a Phosphoimager cassette (Molecular Dynamics) and developed.

VIII. Complementary Polynucleotides

Sequences complementary to the REC-encoding sequences, or any parts thereof, are used to detect, decrease, or inhibit expression of naturally occurring REC. Although use of oligonucleotides comprising from about 15 to 30 base pairs is described, essentially the same procedure is used with smaller or with larger sequence fragments. Appropriate oligonucleotides are designed using OLIGO 4.06 software and the coding sequence of REC. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the REC-encoding transcript.

IX. Expression of REC

Expression and purification of REC is achieved using bacterial or virus-based expression systems. For expression of REC in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express REC upon induction with isopropyl beta-D-thiogalactopyranoside (IPTG). Expression of REC in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding REC by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, REC is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Pharmacia, Piscataway, N.J.). Following purification, the GST moiety can be proteolytically cleaved from REC at specifically engineered sites. FLAG, an 8-amino acid peptide, enables immunoaffinity purification using commercially available monoclonal and polyclonal anti-FLAG antibodies (Eastman Kodak, Rochester, N.Y.). 6-His, a stretch of six consecutive histidine residues, enables purification on metal-chelate resins (QIAGEN Inc, Chatsworth, Calif.). Methods for protein expression and purification are discussed in Ausubel, F. M. et al. (1995 and periodic supplements) Current Protocols in Molecular Biology, John Wiley & Sons, New York, N.Y., ch 10, 16. Purified REC obtained by these methods can be used directly in the following activity assay.

X. Demonstration of REC Activity

An assay for REC activity is based on a prototypical assay for ligand-receptor activity. This assay measures the stimulation of DNA synthesis in Swiss mouse 3T3 cells. A plasmid containing polynucleotides encoding REC are added to quiescent 3T3 cultured cells using transfection methods well known in the art and the transfected cells are then incubated in the presence of [³H]thymidine, a radioactive DNA precursor. Varying amounts of REC ligand are then added to the cultured cells. Incorporation of [³H]thymidine into acid-precipitable DNA is measured over an appropriate time interval using a radioisotope counter, and the amount incorporated is directly proportional to the amount of newly synthesized DNA. A linear dose-response curve over at least a hundred-fold REC ligand concentration range is indicative of receptor activity. One unit of activity per milliliter is defined as the concentration of REC producing a 50% response level, where 100% represents maximal incorporation of [3H]thymidine into acid-precipitable DNA. (McKay, I. and Leigh, I., eds. (1993) Growth Factors: A Practical Approach, Oxford University Press, New York, N.Y., page 73.)

XI. Functional Assays

REC function is assessed by expressing the sequences encoding REC at physiologically elevated levels in mammalian cell culture systems. cDNA is subcloned into a mammalian expression vector containing a strong promoter that drives high levels of cDNA expression. Vectors of choice include. pCMV SPORT (Life Technologies) and pCR 3.1 (Invitrogen, Carlsbad Calif.) both of which contain the cytomegalovirus promoter. 5-10 μl of recombinant vector are transiently transfected into a human cell line, preferably of endothelial or hematopoietic origin, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP) (Clontech, Palo Alto, Calif.), CD64, or a CD64-GFP fusion protein. Flow cytometry (FCM), an automated, laser optics-based technique, is used to identify transfected cells expressing GFP or CD64-GFP, and to evaluate properties, for example, their apoptotic state. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry; Oxford, New York, N.Y.

The influence of REC on gene expression can be assessed using highly purified populations of cells transfected with sequences encoding REC and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success, N.Y.). mRNA can be purified from the cells using methods well known by those of skill in the art. Expression of mRNA encoding REC and other genes of interest can be analyzed by Northern analysis or microarray techniques.

XII. Production of REC Specific Antibodies

REC substantially purified using polyacrylamide gel electrophoresis (PAGE)(see, e.g., Harrington, M. G. (1990) Methods Enzymol. 182:488-495), or other purification techniques, is used to immunize rabbits and to produce antibodies using standard protocols.

Alternatively, the REC amino acid sequence is analyzed using LASERGENE NAVIGATOR software (DNASTAR Inc.) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel supra, ch. 11.) Typically, oligopeptides 15 residues in length are synthesized using an Applied Biosystems Peptide Synthesizer Model 431A using fmoc-chemistry and coupled to KLH (Sigma, St. Louis, Mo.) by reaction with N-maleimidobenzoyl-N-hydroxysuccinimide ester (MBS) to increase immunogenicity. (See, e.g., Ausubel supra.) Rabbits are immunized with the oligopeptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG.

XIII. Purification of Naturally Occurring REC Using Specific Antibodies

Naturally occurring or recombinant REC is substantially purified by immunoaffinity chromatography using antibodies specific for REC. An immunoaffinity column is constructed by covalently coupling anti-REC antibody to an activated chromatographic resin, such as CNBr-activated Sepharose (Pharmacia & Upjohn). After the coupling, the resin is blocked and washed according to the manufacturer's instructions. Media containing REC are passed over the immunoaffinity column, and the column is washed under conditions that allow the preferential absorbance of REC (e.g., high ionic strength buffers in the presence of detergent). The column is eluted under conditions that disrupt antibody/REC binding (e.g., a buffer of pH 2 to pH 3, or a high concentration of a chaotrope, such as urea or thiocyanate ion), and REC is collected.

XIV. Identification of Molecules Which Interact with REC

REC, or biologically active fragments thereof, are labeled with ¹²⁵I Bolton-Hunter reagent. (See, e.g., Bolton et al. (1973) Biochem. J. 133:529.) Candidate molecules previously arrayed in the wells of a multi-well plate are incubated with the labeled REC, washed, and any wells with labeled REC complex are assayed. Data obtained using different concentrations of REC are used to calculate values for the number, affinity, and association of REC with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A substantially purified polypeptide comprising an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, and fragments thereof.

2. A substantially purified variant having at least 90% amino acid identity to the amino acid sequence of claim 1.

3. An isolated and purified polynucleotide encoding the polypeptide of claim 1.

4. An isolated and purified polynucleotide variant having at least 90% polynucleotide sequence identity to the polynucleotide of claim 3.

5. An isolated and purified polynucleotide which hybridizes under stringent conditions to the polynucleotide of claim 3.

6. An isolated and purified polynucleotide having a sequence which is complementary to the polynucleotide sequence of claim 3.

7. An isolated and purified polynucleotide comprising a polynucleotide sequence selected from the group consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and fragments thereof.

8. An isolated and purified polynucleotide variant having at least 90% polynucleotide sequence identity to the polynucleotide of claim 7.

9. An isolated and purified polynucleotide having a sequence which is complementary to the polynucleotide of claim 7.

10. An expression vector comprising at least a fragment of the polynucleotide of claim 3.

11. A host cell comprising the expression vector of claim 10.

12. A method for producing a polypeptide comprising the amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, and fragments thereof, the method comprising the steps of:
a) culturing the host cell of claim 11 under conditions suitable for the expression of the polypeptide; and
b) recovering the polypeptide from the host cell culture.

13. A pharmaceutical composition comprising the polypeptide of claim 1 in conjunction with a suitable pharmaceutical carrier.

14. A purified antibody which specifically binds to the polypeptide of claim 1.

15. A purified agonist of the polypeptide of claim 1.

16. A purified antagonist of the polypeptide of claim 1.

17. A method for treating or preventing a neoplastic disorder, the method comprising administering to a subject in need of such treatment an effective amount of the antagonist of claim 16.

18. A method for treating or preventing an immunological disorder, the method comprising administering to a subject in need of such treatment an effective amount of the antagonist of claim 16.

19. A method for treating or preventing a reproductive disorder, the method comprising administering to a subject in need of such treatment an effective amount of the antagonist of claim 16.

20. A method for treating or preventing a gastrointestinal disorder, the method comprising administering to a subject in need of such treatment an effective amount of the antagonist of claim 16.

21. A method for treating or preventing a nervous disorder, the method comprising administering to a subject in need of such treatment an effective amount of the antagonist of claim 16.

22. A method for treating or preventing a smooth muscle disorder, the method comprising administering to a subject in need of such treatment an effective amount of the antagonist of claim 16.

23. A method for treating or preventing a musculoskeletal disorder, the method comprising administering to a subject in need of such treatment an effective amount of the antagonist of claim 16.

24. A method for detecting a polynucleotide encoding the polypeptide comprising the amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO: 14, SEQ ID NO: 15, SEQ ID NO: 16, and fragments thereof in a biological sample, the method comprising the steps of:
(a) hybridizing the polynucleotide of claim 6 to at least one of the nucleic acids in the biological sample, thereby forming a hybridization complex; and
(b) detecting the hybridization complex, wherein the presence of the hybridization complex correlates with the presence of the polynucleotide encoding the polypeptide in the biological sample.

25. The method of claim 24 wherein the nucleic acids of the biological sample are amplified by the polymerase chain reaction prior to the hybridizing step.
